(19) United States
(12) Patent Application Publication
(10) Pub. No.: US 2026/0038686 A1
(43) Pub. Date: Feb. 5, 2026

(54) AI-BASED TOOL FOR MENTAL HEALTH SUPPORT UTILIZING LARGE LANGUAGE MODELS AND PERSONALIZED RECOMMENDATION

(71) Applicant: Yujia WANG, Irvine, CA (US)

(72) Inventor: Yujia WANG, Irvine, CA (US)

(21) Appl. No.: 19/282,568

(22) Filed: Jul. 28, 2025

Related U.S. Application Data
(60) Provisional application No. 63/678,918, filed on Aug. 2, 2024.

(51) Int. Cl. G16H 50/20; G16H 10/20; G16H 10/60; G16H 20/70; G16H 70/20

(52) U.S. Cl. CPC G16H 50/20; G16H 10/20; G16H 10/60; G16H 20/70; G16H 70/20

(57) ABSTRACT

A method is provided for online mental health support using an AI-based system including an orchestration AI agent and a plurality of vertical expert AI agents, each designed for a specific domain capability. The method includes obtaining user input information from a user; processing the user input information to generate a list of subtasks for responding to the user; based on relevance scores of the vertical expert AI agents, assigning the list of subtasks to the vertical expert AI agents with each subtask assigned a most suitable vertical expert AI agent, where a relevance score of a vertical expert AI agent reflects a degree of relevance between a subtask and the specific domain capability of the vertical expert AI agent; collecting responses from the vertical expert AI agents; and synthesizing the responses from the vertical expert AI agents to create a final message personalized to the user input information.

### CROSS-REFERENCE TO RELATED APPLICATIONS

The present application claims priority to U.S. provisional patent application No., 63/678,918, filed on Aug. 2, 2024, titled “AI-Based Tool For Mental Health Support Utilizing Large Language Models and Personalized Recommendation”, entire content of which is incorporated herein by reference.

### TECHNICAL FIELD

The present disclosure relates to the technical field of artificial intelligence (AI) applications and, more specifically, to a method and system for AI-based mental health assistant utilizing large language models (LLMs) and personalized recommendation.

### BACKGROUND

Existing solutions for providing mental health services have many challenges. For example, there often is a high cost associated with providing cares for the mental, emotional, and spiritual health of a patient. Further, there often is a limited accessibility to people who need such services. Millions of people experience stress and anxiety but lack immediate assistance when they need the professional help. In addition, there often is a privacy concern for patients seeking help. Many people are hesitant to seek help due to their concerns about their privacy, the stigma associated with mental health issues, social point of view, and job security. Moreover, traditional methods often lack the adaptive and responsive nature necessary for effective mental health support, particularly in real-time support.

On the other hand, when using general purpose AI to help mental health services, such general purpose AI often can only provide mental health support at a minimal level due to lack of mental health domain knowledge and efficient knowledge processing engines. For example, existing chatbots often lack the psychological and mental health domain expertise.

The disclosed methods and systems are directed to solve one or more problems set forth above and other problems.

### SUMMARY

A solution architecture is proposed that integrates large language models, vertical AI agent frameworks, and domain-specific intelligence systems to provide adaptive, affordable, and context-aware mental health support at scale.

An aspect of the present disclosure includes a method for providing online mental health support using an AI-based system including an orchestration AI agent and a plurality of vertical expert AI agents in a vertical expert AI agent pool, each expert AI agent being designed for a specific domain capability. The method includes: obtaining user input information from a user interface of a user device inputted by a user; processing the user input information to generate a list of subtasks for responding to the user input information; based on relevance scores of the plurality of vertical expert AI agents, assigning the list of subtasks to the plurality of vertical expert AI agents with each subtask assigned a most suitable vertical expert AI agent, where a relevance score of a vertical expert AI agent reflects a degree of relevance between a subtask and the specific domain capability of the vertical expert AI agent; collecting responses from the plurality of vertical expert AI agents; and synthesizing the responses from the plurality of vertical expert AI agents to create a final message personalized to the user input information, wherein the user input information includes both explicit information and implicit information.

Another aspect of the present disclosure includes a non-transitory computer-readable storage medium containing computer-executable instructions for, when executed by one or more processors, performing a method for providing online mental health support using an AI-based system. The AI-based system includes an orchestration AI agent and a plurality of vertical expert AI agents in a vertical expert AI agent pool, each expert AI agent being designed for a specific domain capability. The method includes: obtaining user input information from a user interface of a user device inputted by a user; processing the user input information to generate a list of subtasks for responding to the user input information; based on relevance scores of the plurality of vertical expert AI agents, assigning the list of subtasks to the plurality of vertical expert AI agents with each subtask assigned a most suitable vertical expert AI agent, where a relevance score of a vertical expert AI agent reflects a degree of relevance between a subtask and the specific domain capability of the vertical expert AI agent; collecting responses from the plurality of vertical expert AI agents; and synthesizing the responses from the plurality of vertical expert AI agents to create a final message personalized to the user input information, wherein the user input information includes both explicit information and implicit information.

Other aspects of the present disclosure can be understood by those skilled in the art in light of the description, the claims, and the drawings of the present disclosure.

### BRIEF DESCRIPTION OF THE DRAWINGS

The foregoing and/or additional aspects and advantages of the present invention will become apparent and comprehensible in the description of the embodiments made with reference to the following accompanying drawings.

FIG. 1 illustrates a personalized AI-based mental health support system according to an embodiment of the present disclosure;

FIG. 2A illustrates certain configuration details of the system according to an embodiment of the present disclosure;

FIG. 2B illustrates a computer system according to an embodiment of the present disclosure;

FIG. 3A illustrates a software structure of the AI-based mental health support system according to an embodiment of the present disclosure;

FIG. 3B illustrates another software structure of the system according to an embodiment of the present disclosure;

FIG. 4 illustrates a user interface of the AI system (named BlissBot) designed for AI-based mental and emotional support according to an embodiment of the present disclosure;

FIG. 5 illustrates another user interface architecture within the BlissBot system according to an embodiment of the present disclosure;

FIG. 6 illustrates a transformer-based neural network architecture utilized by the present disclosure;

FIG. 7 illustrates a process for converting structured mental health knowledge into a machine-readable vector format according to an embodiment of the present disclosure;

FIG. 8 illustrates a block diagram of the embedding generator network for transforming mental health content according to an embodiment of the present disclosure;

FIG. 9 illustrates a prompt-driven vertical retrieval and reasoning framework according to an embodiment of the present disclosure;

FIG. 10 illustrates a vertical AI agent framework according to an embodiment of the present disclosure;

FIG. 11 illustrates an implicit symptom detection subsystem according to an embodiment of the present disclosure;

FIG. 12 illustrates a relevance-driven routing subsystem according to an embodiment of the present disclosure;

FIG. 13 illustrates a flow diagram of a user-driven assessment initiation process according to the present disclosure;

FIG. 14 shows the interactive diagnostic module interface according to an embodiment of the present disclosure;

FIG. 15 illustrates a diagnostic results interface according to an embodiment of the present disclosure;

FIG. 16 illustrates a flow diagram of an adaptive interaction process according to an embodiment of the present disclosure;

FIG. 17 illustrates a user interface of a mental and psychological profile (MPP) engine according to an embodiment of the present disclosure;

FIG. 18 illustrates a psychological factor decomposition view according to an embodiment of the present disclosure;

FIG. 19 shows an adaptive questionnaire user interface according to an embodiment of the present disclosure;

FIG. 20 illustrates a personalized recommendation process according to an embodiment of the present disclosure;

FIG. 21 illustrates a flow diagram of a process for analyzing high priority topics according to an embodiment of the present disclosure;

FIG. 22 illustrates a flow diagram of particular dimension processing according to an embodiment of the present disclosure;

FIG. 23 illustrates the computational formula used to calculate content relevance scores, integrating recovery likelihood, user affinity, and topic alignment factors;

FIG. 24 illustrates a collaborative filtering framework according to an embodiment of the present disclosure;

FIG. 25 illustrates a privacy setting interface according to an embodiment of the present disclosure;

FIG. 26 illustrates a system-level architecture diagram of data management and usage according to an embodiment of the present disclosure;

FIG. 27 illustrates a process for privacy-preserving data according to an embodiment of the present disclosure; and

FIG. 28 illustrates an internal role-based access control (RBAC) framework according to an embodiment of the present disclosure.

### DETAILED DESCRIPTION

To make the objectives, technical solutions, and advantages of the present disclosure more apparent and clearer, the following describes certain embodiments of the present disclosure in further detail with reference to the accompanying drawings. It should be understood that specific embodiments described therein are merely used for explaining the present disclosure instead of limiting the present disclosure.

Mental health support has a critical need in today's society, with many individuals facing stress, anxiety, and other mental health challenges. Traditional mental health services, often suffer from drawbacks including high costs, limited accessibility, and privacy concerns. Accordingly, an AI-based mobile application is provided to provide customized emotional and mental support to its users. This mobile application explores an innovative system that leverages large language models (LLMs) and vertical AI agent framework to provide comprehensive mental health diagnosis, treatment, and support through an advanced chatbot interface, providing a 24/7 available accessibility and it's highly affordable for everyone globally.

Before certain embodiments of the present disclosure are described, definitions or brief introductions of certain terms or entities used in the present disclosure are as follows.

Agent: “Agent” is short for “AI agent”, an autonomous software entity that perceives input, makes decisions, and performs actions. In AI systems, agents are responsible for tasks like reasoning, interaction, or recommendation. Each agent is designed to handle specific psychological functions, contributing to modular and intelligent mental health support.

Vertical AI Agent Framework: “Vertical” refers to a specialized domain within a broader field. A Vertical AI Agent Framework is a system where each AI agent is designed for a specific functional domain and operates collaboratively under a central orchestrator. The “vertical” may specifically denote subdomains of mental health, such as anxiety support or emotional regulation, enabling modular and domain-specific psychological assistance.

Orchestration Agent: “orchestration” means organizing different elements to work together harmoniously, like a conductor managing an orchestra. An Orchestration Agent is the central AI component that coordinates multiple specialized agents. It interprets user input, assigns tasks to appropriate mental health expert agents, integrates their outputs, and generates coherent, context-aware responses. It ensures adaptive, multi-agent collaboration within the mental health support system.

Large Language Models (LLMs): large language models are neural network-based AI systems trained on massive text corpora to understand and generate human-like language. Originally developed by leading AI labs (e.g., OpenAI, Google), here LLMs are applied to interpret user input, detect emotional cues, and produce personalized mental health responses.

Hybrid Diagnosis Engine: a hybrid diagnosis engine combines two approaches: implicit analysis and explicit assessment. Implicit modules infer emotional or psychological states from user behavior and language, while explicit modules use standardized questionnaires. Both are integrated to generate a comprehensive, real-time understanding of the user's mental health condition, enabling more accurate and adaptive support.

Implicit Analysis Module: an implicit analysis module passively monitors user behavior, such as tone, word choice, and interaction patterns, to infer emotional or psychological signals without asking direct questions. It helps detect early signs of distress or mental health risk by analyzing free-form input, contributing to real-time, low-friction diagnosis and support.

Explicit Assessment Module: an explicit assessment module administers structured, clinically validated questionnaires—such as PHQ-9 or GAD-7—to evaluate a user's mental health. Unlike passive detection, this method requires user participation. It complements implicit analysis by providing standardized data for diagnosis, ensuring clinical rigor and interpretability in the information collection process.

Mental and Psychological Profile (MPP): the mental and psychological profile (MPP) is a dynamic, multi-dimensional record of a user's emotional, cognitive, and behavioral states. It is continuously updated through system interactions, implicit signals, and explicit assessments. The MPP serves as the foundation for personalization, recommendation, and longitudinal monitoring of mental health progress.

Structured Content: structured content refers to information organized in predefined formats such as databases, tables, forms, or questionnaires. It is easily parsed and processed by machines due to its consistent structure. The structured content includes diagnostic scales (e.g., PHQ-9), medical codes, and predefined symptom taxonomies used for mental health assessment and retrieval.

Unstructured Content: unstructured content is information presented in free-form formats without predefined organization, such as text messages, therapy transcripts, or journal entries. It requires natural language processing to extract meaning. The unstructured content includes user conversations, emotional narratives, and expert-written materials processed into semantic embeddings.

Semantic Knowledge Engine: a semantic knowledge engine transforms structured and unstructured psychological content into machine-readable vector embeddings. It enables real-time, context-aware retrieval of relevant knowledge. It powers the system's ability to deliver accurate, psychologically grounded responses by linking user input to domain-specific therapeutic information.

Vector: a vector is a mathematical object represented as an ordered list of numbers, often used to describe direction and magnitude in space. In machine learning, vectors numerically represent data such as words, sentences, or images. Vectors allow systems to measure similarity, perform calculations, and reason about meaning in a structured, quantitative way.

Vector Embedding: vector embedding is the process of converting text or other unstructured input into a fixed-length numerical vector. These embeddings capture semantic features, such as word meaning and context, and allow AI models to compare, retrieve, and reason over information. The embeddings enable psychological content and user input to be processed semantically.

Domain-Specific Expert Agent: a domain-specific expert agent is an AI module trained or configured to operate within a narrowly defined field, such as anxiety management or emotional regulation. Each agent focuses on a distinct area of mental health, allowing for modular, specialized reasoning and more accurate, personalized support for the user.

Prompt Engineering: prompt engineering is the practice of crafting precise input queries to guide a large language model (LLM) in producing relevant and useful responses. The prompts are structured to incorporate context from user input, historical interactions, and psychological profiles, enabling the AI to generate responses that are aligned with the user's mental and emotional needs.

Retrieval-Augmented Generation (RAG): retrieval-augmented generation (RAG) is an AI technique that enhances response quality by retrieving external information before generating text. Unlike standard language models, RAG fetches relevant content, such as psychological documents or prior user data, at runtime and incorporates it into the prompt. In this invention, RAG enables the system to produce more informed, personalized, and context-aware mental health responses.

Knowledge Vector Database: a knowledge vector database stores information in the form of vector embeddings, allowing semantic search rather than keyword matching. It contains vectorized psychological content, such as therapy frameworks and assessment data, enabling the system to retrieve contextually relevant knowledge and generate meaningful mental health support responses.

Personalization Engine: personalization refers to adapting a system's behavior to fit individual user needs based on their data and behavior. A personalization engine automates this process. In this invention, the engine uses the user's mental and psychological profile (MPP) and interaction history to adjust tone, timing, and content, delivering mental health support that is continuously aligned with the user's evolving emotional and psychological state.

Contextual Prompt Generator: a contextual prompt generator creates structured input prompts for language models by incorporating relevant context, such as user history, emotional signals, or recent interactions. It transforms raw user input into enriched prompts that help AI agents generate responses that are emotionally attuned, situationally aware, and psychologically appropriate.

Dynamic Profiles: dynamic profiles are continuously updated representations of a user's mental, emotional, or behavioral state. Unlike static records, they evolve in real time based on new data and interactions. In this invention, dynamic profiles reflect the user's changing psychological conditions and guide personalized recommendations, adaptive responses, and longitudinal mental health tracking.

Symptom Score Aggregator: a symptom score aggregator compiles and calculates scores based on detected psychological symptoms, such as sadness or anxiety, identified in user input. It evaluates multiple signals from implicit analysis, assigns weighted values, and produces a cumulative score used to determine whether intervention or further assessment is needed.

Threshold Evaluation Module: a threshold evaluation module determines whether a computed score, such as a symptom severity score, exceeds a predefined limit that warrants action. It is used to trigger alerts or escalate support when a user's psychological signals cross certain thresholds, helping ensure timely and appropriate mental health responses.

User Similarity Network: a user similarity network groups users based on shared psychological traits, behavior patterns, or engagement histories. It is used to identify users with comparable mental health profiles, enabling collaborative filtering, personalized recommendations, and content propagation based on what has helped similar users.

Collaborative Filtering: collaborative filtering is a recommendation technique that suggests content to a user based on the preferences or outcomes of similar users. It helps identify effective mental health resources by analyzing patterns across users with comparable profiles, enhancing personalization through community-driven intelligence.

Content Relevance: content relevance refers to how well a piece of content aligns with a user's current needs, preferences, or psychological state. The relevance is determined by comparing content metadata and vector embeddings with the user's dynamic profile, ensuring that recommended resources are timely, personalized, and psychologically appropriate.

The tf-idf-method: the tf-idf method stands for term frequency-inverse document frequency. “TF” measures how often a word appears in a specific document. “IDF” reduces the importance of words that appear in many documents. Together, they highlight words that are frequent and unique. A similar method is used to score content: items that are both helpful and rare among similar users get higher priority in recommendations, improving personalization and relevance.

Agent Relevance Evaluation Module: the agent relevance evaluation module scores how well each AI agent matches a specific user input or task. It compares the user's message with each agent's domain expertise, such as anxiety or emotional regulation, and assigns a relevance score. This ensures that the most suitable agent is selected to respond.

Priority Agent Selector: a priority agent selector is a decision module that ranks and selects the best-suited AI agent for a given task, based on relevance, context, and urgency. It is used to route user input to the most appropriate mental health agent, such as one focused on anxiety or self-esteem, ensuring that support is precise, responsive, and psychologically aligned.

Masked Identifier (MID): a masked identifier (MID) is a unique code that replaces a user's real identity during data processing to protect privacy. MIDs are used to anonymize sensitive mental health data, allowing the system to analyze and personalize support without exposing personally identifiable information.

Tiered Access Control: tiered access control is a security framework that restricts data access based on user roles, responsibilities, or sensitivity levels. The mental health data is separated into layers, from anonymized statistics to identity-linked records, and only authorized personnel can access each tier, ensuring strong protection of user privacy and regulatory compliance.

User-Defined Data Governance: user-defined data governance allows individuals to control how their personal data is collected, stored, and shared. The users can set consent levels for mental health data, choosing whether it's retained, anonymized, or shared with experts or for research, ensuring transparency, autonomy, and alignment with privacy preferences.

Anonymized Health Data Layer: an anonymized health data layer contains user health information that has been stripped of personally identifiable details. It enables safe internal use of mental health data, for purposes like system improvement or trend analysis, while ensuring individual identities remain hidden and protected from unauthorized access.

Semantic Retrieval: semantic retrieval is a search technique that finds information based on meaning rather than exact keywords. It uses vector embeddings to match user input with conceptually related content. It enables the system to retrieve relevant psychological resources, such as therapy guides or assessments, even if users express their needs in varied or informal language.

Symptom Evaluation Engine: a symptom evaluation engine analyzes user input, such as language patterns, tone, and pacing, to identify possible mental health symptoms. The engine scans for signs like hopelessness or anxiety and assigns scores based on their presence and intensity. These scores are aggregated across multiple signals to estimate overall symptom severity, which informs downstream modules such as diagnosis or risk alerts.

Human-in-the-Loop Review: human-in-the-loop (HITL) review is a process where human experts oversee, validate, or intervene in AI-driven decisions. It is used to review sensitive outputs, such as recommendations or flagged risk signals, to ensure safety, appropriateness, and quality. HITL adds an extra layer of accountability in mental health scenarios where full automation may not be sufficient.

Role-Based Access Control (RBAC): role-based access control (RBAC) is a security model that restricts system access based on users' roles within an organization. The RBAC limits access to sensitive mental health data and system functions according to assigned roles, such as clinicians, data scientists, or administrators, ensuring compliance with privacy and regulatory requirements.

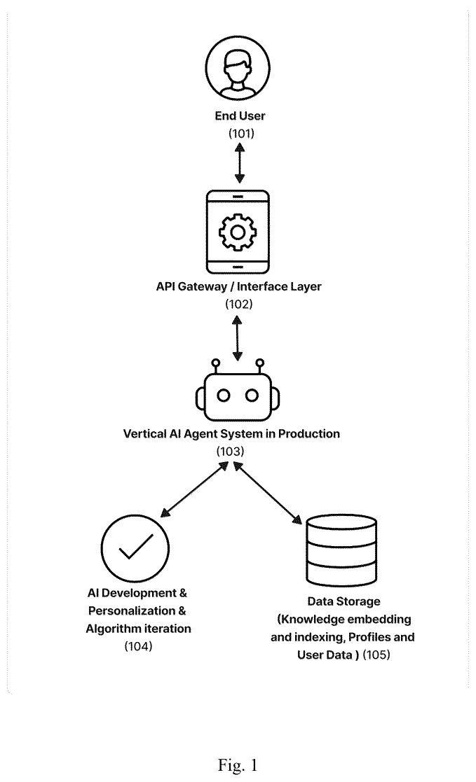

FIG. 1 illustrates a personalized AI-based mental health support system according to an embodiment of the present disclosure. As shown in FIG. 1, the system may include an application program interface (API) gateway 102, a vertical AI agent system 103, an AI development, personalization, and algorithm iteration module 104, and a data storage module 105. Other components may also be included.

The API gateway 102 may include any appropriate computer system configured with software applications, such as a mobile phone, a laptop computer, a tablet, a desktop computer, a server, etc. The API gateway 102 may provide an interface layer for the end user to access the system. The vertical AI agent system 103 may include any server computer configured with AI software trained to implement the AI-based mental health support system, such as one or more server in a standalone configuration or in a cloud configuration. The AI development, personalization, and algorithm iteration module 104 may be a software application running on a same or different server of the vertical AI agent system 103. The data storage module 105 may include any appropriate storage medium or database system coupled to the vertical AI agent system 103. The vertical AI agent system 103 may be dynamic updated or trained and, during user operation, the vertical AI agent system 103 may also be referred as a vertical AI agent system in production.

During operation, an end user 101 may access the system through a web or mobile application via the API gateway 102 (e.g., via the interface layer to allow user access). For example, the end user may provide certain user input to the interface layer of the API gateway, and the API gateway 102 may transmit the user input to the vertical AI agent system 103, which serves as the centralized intelligence engine composed of domain-specific AI agents and orchestration logic. The vertical AI agent system operates in conjunction with AI development, personalization, and algorithm iteration module 104, which performs continuous updates based on user feedback and behavioral data, and the data storage module 105, which retains structured and unstructured information including vertical knowledge, semantic vectors, personalized mental health profiles, and historical interactions. The data flow among those components may be dynamic and bidirectional, so as to enable adaptive reasoning and continuous improvement.

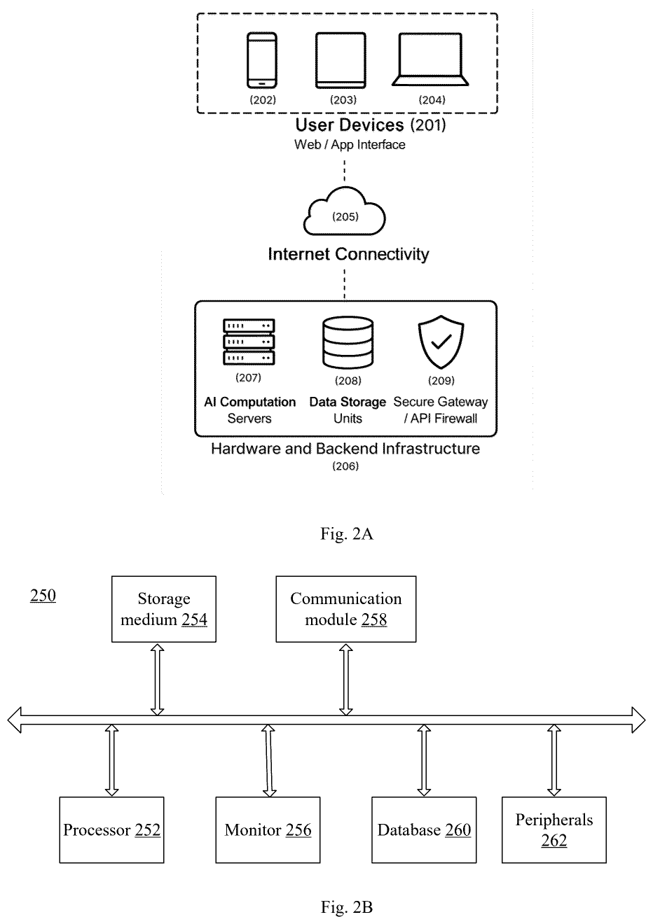

FIG. 2A illustrates certain configuration details of the system according to an embodiment of the present disclosure. As shown in FIG. 2A, the physical architecture of the system may include user devices 201, including for example a smartphone 202), a tablet 203, and/or a laptop 204, etc., which are connected through Internet connectivity 205 to interact with the backend systems. The Internet connectivity 205 may include any wired or wireless communication connections to connect the user devices 201 to the backend systems. Further, the backend infrastructure or backend systems 206 may include multiple backend components, such as AI computation servers 207 for processing large language models and agent workflows, data storage units 208 for storing user profiles, chat logs, and knowledge data, and a secure gateway/API (application programming interface) firewall 209 to manage authentication and encrypted communication. The backend infrastructure 206 may support secure, real-time operations across various user devices and backend components, supporting secure and scalable delivery of AI-based mental health services.

The various user devices, backend systems, servers, and other system may be implemented by any suitable computer system(s). FIG. 2B illustrates a computer system according to an embodiment of the present disclosure. As shown in FIG. 2B, computer system 250 may include a hardware processor 252, storage medium 254, a monitor 256, a communication module 258, a database 260, and peripherals 262. Certain devices may be omitted, and other devices may be included.

Processor 252 may include any appropriate processor or processors. Further, processor 252 can include multiple cores for multi-thread or parallel processing. Storage medium 254 may include memory modules, such as Read-only Memory (ROM), Random Access Memory (RAM), flash memory modules, and erasable and rewritable memory, and mass storages, such as CD-ROM, U-disk, and hard disk, etc. Storage medium 254 may store computer programs for implementing various processes, when executed by processor 252. Monitor 256 may include any appropriate display for displaying data processed by the processor 252, such as an LCD display screen or a touch screen, etc.

Further, peripherals 262 may include I/O devices such as a keyboard and a mouse. Communication module 258 may include network devices for establishing connections through the Internet connectivity 205. Database 260 may include one or more databases for storing certain data and for performing certain operations on the stored data, such as database searching and analysis.

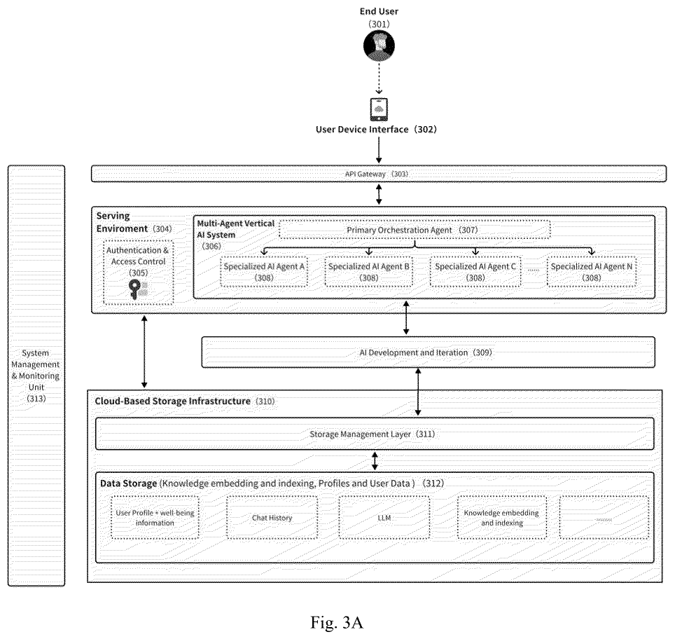

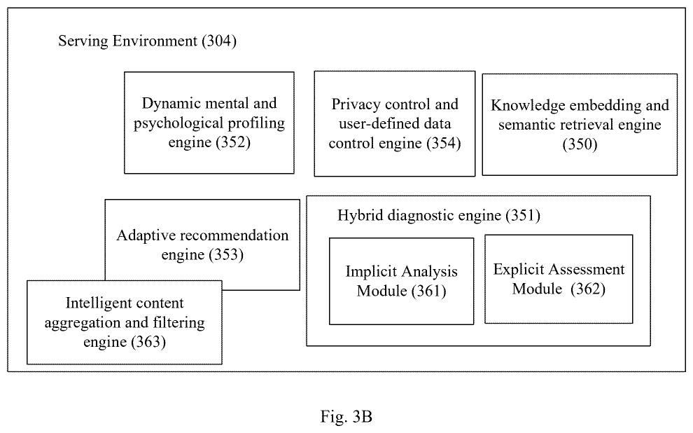

FIG. 3A illustrates a software structure of the AI-based mental health support system according to an embodiment of the present disclosure. The AI-based mental health support system (simply the AI system or the system) includes an API gateway layer 303, and a core serving environment 304. The core serving environment 304 hosts the runtime logic for AI agents of the system and other supporting services. For example, the core serving environment 304 may include an authentication and access control module 305 and a multi-agent vertical AI system 306, which includes a primary orchestration agent 307 coordinating multiple specialized AI agents 308.

Further, the software structure may include a cloud-based storage infrastructure 310. The cloud-based storage infrastructure 310 may include various software components handling, processing, storing, and retrieving related data in the system, including interacting with the user 301 through the user interface and the serving environment, etc. For example, the cloud-based storage infrastructure 310 may include a storage management layer 311 and a centralized data storage module 312. The centralized data storage module 312 may be used to store and/or retrieve structured data relevant to the system's operations, including personalized mental health profiles, longitudinal chat history, large language model components, and vertical psychological knowledge.

An AI development and iteration module 309 may be configured between the multi-agent vertical AI system 306 and the cloud-based storage infrastructure 310, such that the AI system can be dynamically updated or optimized in real time. For example, the AI system's reasoning outputs can be iteratively refined through the AI development and iteration module 309, which can update model weights, fine-tunes logic, and integrates user feedback. Further, the software structure may include a system management and monitoring module 313 for controlling the AI system to ensure operational stability, policy enforcement, and regulatory compliance.

The software structure may also include a user device interface 302, the end user 301 may interact with the AI system through the user device interface 302. User inputs are processed through the API gateway 303, which manages secure communication and traffic routing into the backend. Thus, the user inputs are routed through the API gateway into the serving environment 304 (which may also be referred to backend service, or microservice backend), the central orchestration bot coordinates multiple specialized AI agents to process tasks. The system includes modules for login, chatbot management, and connects to a cloud database storing user profiles, chat history, LLMs, and psychological knowledge. A system management layer oversees the entire workflow. Accordingly, this software architecture may support real-time inference, personalized interaction, and scalable deployment across diverse use cases.

The above described modules and components may be worked together or integrated to provide AI-based mental health support services. That is, the system includes a set of functionally distinct but architecturally integrated modules that together enable advanced mental health support, emotional inference, and therapeutic recommendation. These modules collectively realize a technical framework that is modular, scalable, and adaptable across use cases requiring domain-specific AI reasoning.

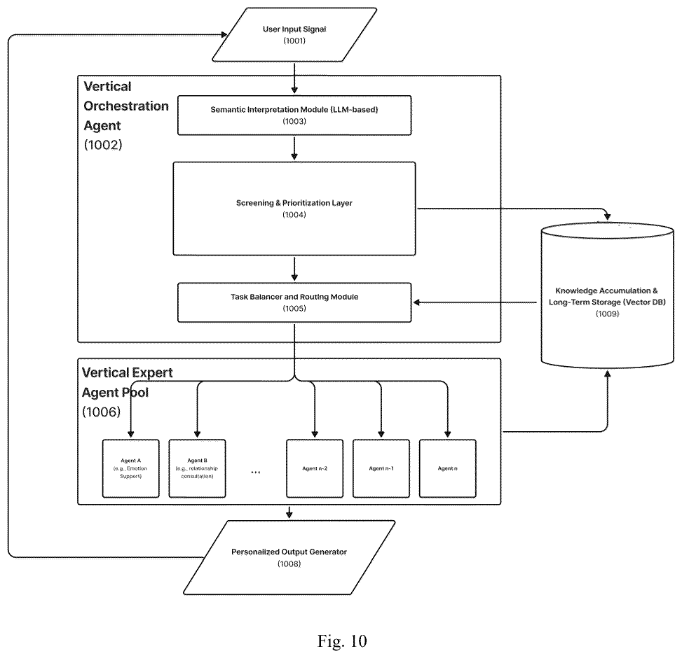

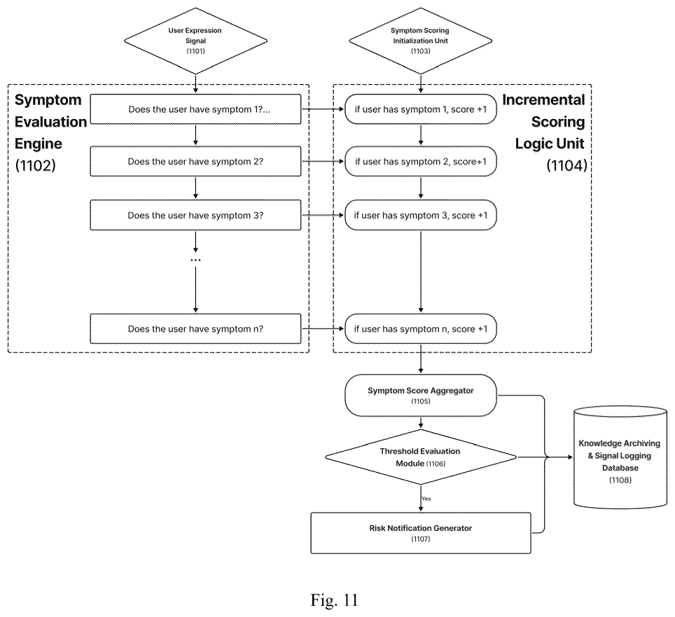

For example, to illustrate the multi-agent vertical AI system 306, FIG. 10 shows certain details of a vertical AI agent framework according to an embodiment of the present disclosure. The framework may use a coordinator agent, semantic interpretation module, screening and prioritization layer, routing module, and a pool of domain-specific expert agents. As such, the framework may enable modular task assignment and collaborative reasoning across specialized AI agents to generate personalized mental health support.

More specifically, as shown in FIG. 10, the vertical AI agent framework may include vertical orchestration agent 1002 and vertical expert agent pool 1006, etc. The vertical expert agent pool 1006 includes a plurality of vertical expert AI agents, and each expert AI agent may be designed or trained for a specific domain capability. In operation, the process of the framework is initiated when a user input signal 1001 is received by a central coordination unit termed the vertical orchestration agent 1002 (i.e., a coordinator node). This agent is responsible for managing the workflow and initiating a series of cognitive processing steps.

The user input signal or simply input may be first interpreted by the semantic interpretation module (LLM-based) 1003, which decodes user intent and extracts contextual meaning. The interpreted input is then passed to the screening & prioritization layer 1004, which evaluates the input for indicators of psychological or emotional concerns. This layer enables early detection of risk factors and flags the input for specialized handling if risk factors are detected.

Subsequently, the task balancer and routing module 1005 may determine the appropriate downstream agents to engage. The task balancer and routing module 1005 interfaces with the vertical expert agent pool 1006, a dynamic set of specialized AI agents each with a specific domain capability (e.g., Agent A for emotion support, Agent B for relationship guidance, and so on), each trained or configured for a narrow, domain-specific task. That is, each expert agent is vertically specialized, for example, in domains such as emotional regulation, cognitive restructuring, anxiety management, or spiritual support, and responds independently to user needs, while contributing collaboratively to system-wide decision-making through the orchestration agent 1002.

These specialized or expert agents can process the assigned tasks or subtasks in parallel or in sequence, depending on the routing strategy. The responses from the specialized agents are collected and synthesized by the personalized output generator 1008, which may create a final message tailored to the user's situation, tone, and support needs. Additionally or simultaneously, selected data (e.g., from the screening & prioritization layer 1004) and responses/outcomes (e.g., from the specialized agents) are forwarded to the knowledge accumulation & long-term storage (Vector DB) 1009, ensuring that learnings from each session contribute to the evolving intelligence of the system and enable continuous personalization. Thus, this framework enables vertical specialization, interpretable reasoning, adaptive safety controls, and scalable delivery of context-aware mental health support, all while maintaining modularity and extensibility across psychological domains. In other words, this framework enables modular, expert-driven processing of mental health support tasks through a multi-stage orchestration pipeline.

At runtime, in some embodiments, a central orchestration agent may perform central coordination by interpreting user intent and routing tasks/subtasks to specialized domain-specific expert agents. That is, the central orchestration agent may process the user inputs to create a list of subtasks or tasks corresponding to the user inputs such that the responses to the user can be generated based on the list of subtasks/tasks. The central orchestration agent may then assign those subtasks/tasks to the individual relevant expert agents. These agents then perform localized reasoning within their vertical domain and return structured outputs. Through this multi-agent collaboration across domain functions, the system generates personalized, psychologically coherent responses that reflect both real-time user signals and domain-specific intelligence.

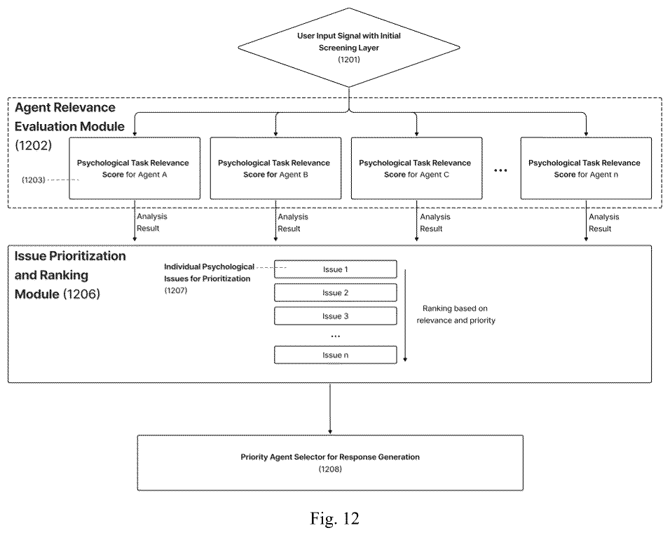

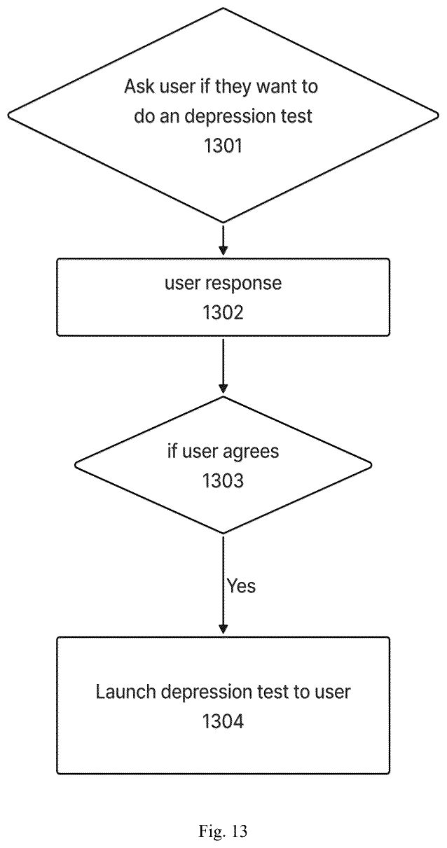

FIG. 12 illustrates a relevance-driven routing subsystem according to an embodiment of the present disclosure. The relevance-driven routing subsystem (e.g., Routing module 1005 in FIG. 10) may provide a relevance-driven routing mechanism in the vertical multi-agent framework. An input signal is processed by a screening layer, after which a set of relevance scores is computed by an agent relevance evaluation module, with individual outputs corresponding to each candidate agent. These scores feed into an issue prioritization and ranking module, where candidate issues are ordered based on relevance and importance. The system then selects the most suitable agent through a priority agent selector to generate a tailored response. In one embodiment, an agent with a highest relevance score may be selected as the most suitable vertical agent.

More specifically, the subsystem incorporates a structured multi-stage pipeline for determining the most contextually appropriate agent response. As shown in FIG. 12, upon receiving a user input signal, the initial screening layer 1201 performs semantic and contextual filtering to normalize the user input signal and identify relevant intent patterns. The filtered user input signal is then processed by an agent relevance evaluation module 1202, which evaluates the alignment of the user input signal against the domain capabilities of multiple vertical agents. Each individual agent receives a psychological task relevance score 1203 (e.g., psychological task relevance score for Agent A, psychological task relevance score for Agent B, . . . , psychological task relevance score for Agent n), reflecting its suitability for handling the given input. A relevance score of an agent may reflect a degree of relevance between a given input (or subtask/task, or an issue, etc.) and the specific domain capability of the vertical agent.

This analysis results from the multiple vertical agents are passed into an issue prioritization and ranking module 1206, which synthesizes the relevance outputs and constructs a priority queue of candidate issues (1207). These issues are sorted based on both content relevance and contextual urgency. The priority agent selector for response generation (1208) then identifies the most relevant and high-priority agent to execute the response, ensuring that the system delivers the most appropriate support action in a scalable and modular manner.

This component may serve as a core decision-making layer within the overall vertical agent framework, enabling intelligent task routing in real time based on agent specialization and problem complexity.

While the illustrated example focuses on psychological task relevance, the architecture of the agent relevance evaluation module and the issue prioritization and ranking module is modular and domain-agnostic. The same mechanism can be adapted for other vertical applications—such as educational tutoring, career coaching, or lifestyle recommendation—by adjusting the scoring logic and issue taxonomy. This allows the framework to serve as a generalized multi-agent coordination engine capable of routing user inputs to the most suitable expert agent across a wide range of task domains.

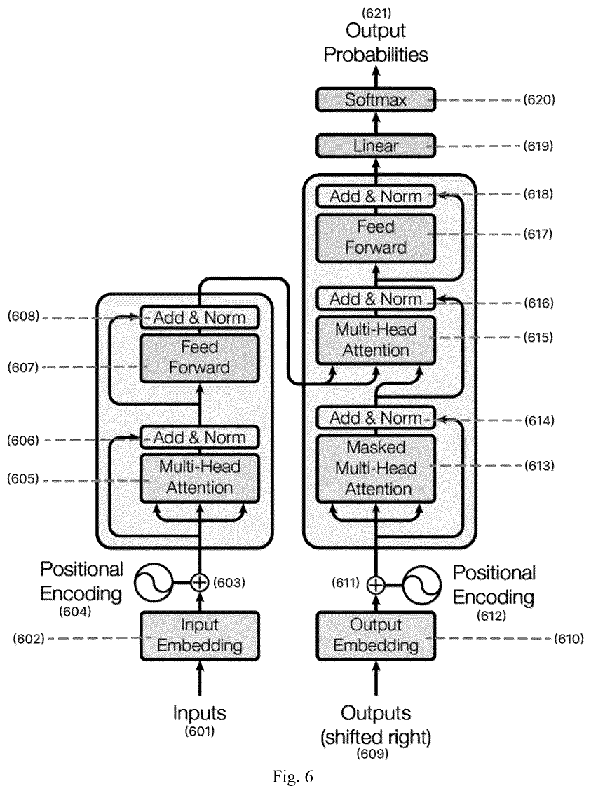

The agents (the orchestration agent and the specialized AI agents) may be AI agents based on LLMs, which is built on a transformer-based neural network architecture. FIG. 6 illustrates a transformer-based neural network architecture utilized by the present disclosure to process user input and generate psychologically relevant output responses. That is, the natural language processing focuses on personalized mental health support.

As shown in FIG. 6, on the encoder side, input tokens 601 are embedded via an input embedding layer 602 and combined with positional encoding 604 at node 603 to preserve sequential structure. These embeddings are processed by a series of multi-head attention modules 605, add-and-normalization layers 606, and feedforward layers 607, followed by another normalization stage 608, producing contextualized representations of the user's input.

The decoder receives shifted output tokens 609, which are embedded 610, then augmented with positional encodings 612 at point 611. The decoder comprises masked multi-head attention 613 to maintain autoregressive behavior, followed by normalization 614, encoder-decoder attention 615, additional normalization 616, and a feedforward transformation 617, finalized by a third normalization layer 618. The resulting vectors are linearly projected 619, transformed into output probabilities via a softmax layer 620, and presented as predicted tokens 621.

Returning to FIG. 3A, the AI development and iteration 309 may also update the runtime LLM-based AI Agents, as well as training standby LLM-Based AI Agents. In this context, the transformer is integrated into the agent framework to enable high-dimensional emotional reasoning, user-specific context alignment, and real-time generation of empathetic and coherent support responses.

The platform develops vertically specialized AI agents through a range of techniques that include, but are not limited to, model training, fine-tuning on domain-specific data, retrieval-augmented generation (RAG), and prompt engineering. These methods are used independently or in combination depending on the agent's functional scope and deployment context. Each agent is aligned to a specific psychological domain and operates under the coordination of a central orchestration engine, enabling personalized, context-aware support grounded in both learned and retrieved domain knowledge.

In addition to leveraging public foundation models (e.g., LLMs), the system can train its own language models using curated psychological datasets. The training pipeline includes: data collection & preprocessing, which ingests anonymized therapy transcripts, emotional assessments, and user conversations, cleans and tokenizes them; supervised training, which applies mental health supervision signals to fine-tune LLMs on conversation structure, therapeutic techniques, and clinical correctness; and continual fine-tuning, uses live interaction data to improve inference quality and adjust model weights responsively.

Accordingly, comparing with monolithic chatbots/agents, the disclosed vertical multi-agent architecture offers certain advantages. For example, as agents are constructed around psychological subdomains and operate with independent logic chains, enhancing responsiveness and interpretability, having decentralized modularity with domain fidelity. Further, as the orchestration agent dynamically delegates tasks, routes outputs, and synthesizes response plans, functioning as the central decision node for context-sensitive dialogue flows and therapeutic path selection, providing orchestration-orchestrated adaptive reasoning.

The multi-agent vertical AI system may include or be used to implement various subsystems or functional modules for various services provided by the system. FIG. 3B illustrates certain subsystems of the multi-agent vertical AI system according to an embodiment of the present disclosure. As shown in FIG. 3B, the system may include a knowledge embedding and semantic retrieval engine 350, a hybrid diagnosis engine 351, a dynamic mental and psychological profiling (MPP) engine 352, an adaptive recommendation engine 353, and a privacy control and user-defined data control engine 354, etc. Othe subsystems may also be implemented and/or included.

The term “engine” may refer to a software module or component to provide certain functionalities. The various components shown in FIG. 3B may also be based on LLMs or other logics. Further, the multiple AI agents shown in FIG. 3A may operate independently from the multiple AI agents shown in FIG. 3A, or may work with the multiple AI agents shown in FIG. 3A, or may be integrated into or implemented by the multiple AI agents shown in FIG. 3A. For example, the knowledge embedding and semantic retrieval engine 350 and/or the privacy control and user-defined data control engine 354 may operate independently; the dynamic mental and psychological profiling (MPP) engine 352 may operate parallelly; and the hybrid diagnosis engine 351 and the adaptive recommendation engine 353 may work independently or be integrated in the multiple AI agents shown in FIG. 3A.

To enable effective knowledge utilization, the knowledge embedding and semantic retrieval engine 350 processes heterogeneous psychological content, including evidence-based therapy guides, structured diagnostic frameworks (e.g., DSM-5), and anonymized conversation logs, transforming them into vectorized representations for machine reasoning. These embeddings are stored in a domain-specific vector database that supports semantic similarity queries and real-time contextual retrieval (e.g., Data Storage 310 in FIG. 3). Structured domain knowledge, such as CBT guidelines, psychoeducation materials, and psychometric scoring systems, may be embedded into vector space representations. During user interaction, this knowledge vector database enables real-time retrieval based on semantic relevance to current user queries. Retrieval operations support contextual chaining, allowing multiple documents or embeddings to inform a single therapeutic response.

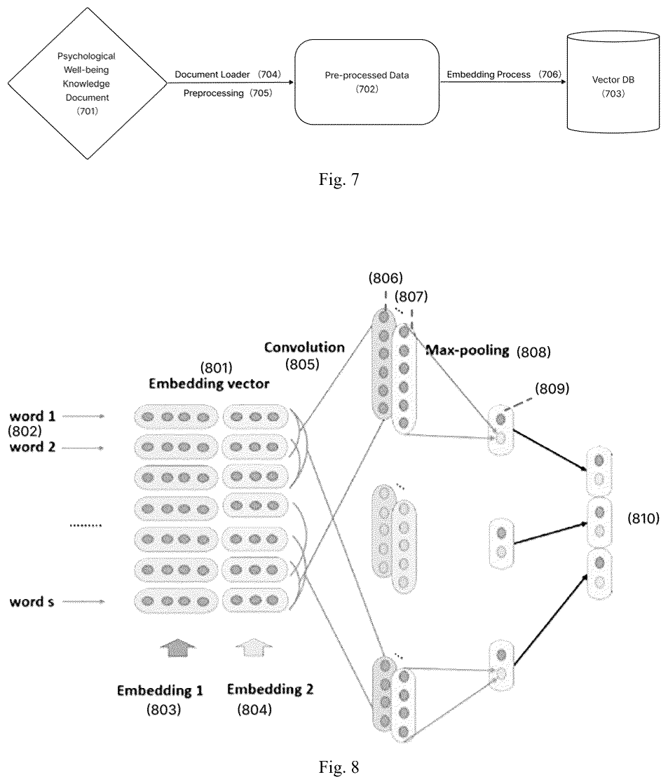

FIG. 7 illustrates a process for converting structured mental health knowledge into a machine-readable vector format according to an embodiment of the present disclosure. The converting process may include a knowledge embedding process designed to convert psychological well-being documents into machine-understandable vector representations that can be accessed in real-time by the AI support system. A raw knowledge source 701, such as therapy guidelines, mental health assessments, or curated expert content, is first ingested via a document loader 704. After the content is ingested, structured preprocessing 705 is performed on the content, including cleaning, segmentation, and formatting, to generate standardized textual data 702. This pre-processed content is then passed through an embedding process 706, which transforms the content into dense vector representations stored in a vector database 703. This process may enable high-performance semantic retrieval across mental health topics, allowing the system to support adaptive, contextualized reasoning in user interactions.

FIG. 8 illustrates a block diagram of the embedding generator network for transforming mental health content according to an embodiment of the present disclosure. As an optional feature, the knowledge embedding pipeline in FIG. 7 may be enhanced using the embedding generator network, which may have convolutional neural network (CNN)-based architectures. As shown in FIG. 8, after textual content has been embedded into token-level vectors, as described in FIG. 7, these vectors can be processed to extract deeper semantic and syntactic features. More specifically, word tokens 802 are embedded into vectors 801 using either the static embedding 803 (Embedding 1) or the contextual embedding 804 (Embedding 2). These embedding vectors are passed through convolutional layers 805, which apply multiple filters to capture n-gram patterns and local structures. The resulting feature maps 806, with per-kernel activations 807, are pooled via max-pooling operations 808 to extract the most salient representations 809. The most salient representations 809 are aggregated into a final output feature set 810 that can enhance downstream modules, such as emotion recognition, stress detection, and personalized intent modeling, within the vertical AI framework.

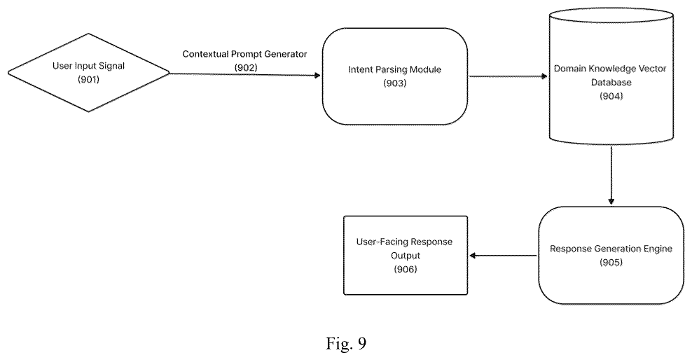

Based on the processes in FIG. 7 and FIG. 8, FIG. 9 illustrates a prompt-driven vertical retrieval and reasoning framework according to an embodiment of the present disclosure. In one embodiment, as shown in FIG. 9, a user input signal is parsed by a contextual prompt generator, triggering a multi-stage inference process involving an intent parsing module, a domain knowledge vector database, and a response generation engine to produce a user-facing response output. In certain embodiments, the framework in FIG. 9 may build upon the knowledge processing pipeline introduced in FIG. 7 and FIG. 8 by utilizing the embedded domain knowledge during real-time user interaction, as a prompt-driven vertical retrieval and reasoning framework that bridges raw user input with expert-informed response generation.

Specifically, a user input signal 901, such as a user message or inquiry, is received via the front-end interface (e.g., the user device interface). The user input signal 901 is converted into a structured semantic prompt by a contextual prompt generator 902, which incorporates factors such as user intent, emotional cues, and conversation history, etc. The generated structured semantic prompt is then passed into an intent parsing module 903, which may be implemented via a lightweight or task-specific large language model, to interpret the prompt and formulate a targeted semantic query.

The targeted semantic query may be used to retrieve relevant content from the domain knowledge vector database 904, which may be constructed through the embedding and convolutional processes shown in FIG. 7 and FIG. 8. The retrieved domain knowledge vector is then passed to a downstream response generation engine 905, where a full-scale LLM generates a personalized, psychologically coherent output as the result in response to the user input signal 901. The result is delivered back to the user through the user-facing response output 906, completing the closed-loop interaction with domain-specific relevance and emotional alignment. This vertical integration of user prompts, intent parsing, and expert-informed generation enables the system to deliver scalable, adaptive, and context-aware mental health support at a depth and specificity not possible with general-purpose chatbot architectures.

Together, the processes and functional modules shown above may support improved semantic reasoning, allow the system to dynamically query relevant information, extract latent user needs, and deliver high-relevance AI responses, enabling intelligent, modular, and context-aware support for mental and emotional health scenarios.

Accordingly, the knowledge embedding and semantic retrieval engine 350 transforms structured and unstructured psychological materials, including validated therapeutic frameworks, clinical assessment protocols, and anonymized interaction transcripts—into high-dimensional vector embeddings. These embeddings are indexed within a machine-readable semantic database, optimized for real-time retrieval based on user input and system context.

In parallel, the system deploys a vertically modular AI agent ecosystem, where each agent is trained or adapted to a particular mental health function such as anxiety regulation, cognitive restructuring, or emotional validation. The development of these agents may involve, but is not limited to, foundational model training, domain-specific fine-tuning, retrieval-augmented generation (RAG), and prompt engineering. These agents are capable of delivering both generative and knowledge-grounded responses tailored to the user's psychological profile.

At runtime, a central orchestration agent governs orchestration by interpreting user intent and routing tasks to specialized domain-specific expert agents. These agents then perform localized reasoning within their vertical domain and return structured outputs. Through this multi-agent collaboration across domain functions, the system generates personalized, psychologically coherent responses that reflect both real-time user signals and domain-specific intelligence. Such architecture ensures response precision, domain interpretability, and system scalability, supporting both real-time user interaction and extensibility across mental, emotional, and behavioral support contexts.

Returning to FIG. 3B, the system includes the hybrid diagnosis engine 351, which is configured to infer the user's psychological and emotional state. The hybrid diagnosis engine 351 may include an implicit analysis module 361 and an explicit assessment module 362. The implicit analysis module 361 applies LLMs and behavioral classifiers to detect psychological signals from free-form user interactions (e.g., implicit information such as linguistic tone, word choice, interaction frequency). The explicit assessment module 362 guides users through structured diagnostic workflows derived from validated scales, such as Patient Health Questionnaire-9 (PHQ-9) or Generalized Anxiety Disorder-7 (GAD-7), and integrates the results into user profiles for longitudinal analysis.

That is, by using the implicit diagnostic and explicit assessment, the hybrid diagnosis engine 351 provides hybrid diagnosis flow or a dual-mode diagnosis flow. The dual-mode diagnosis capability enables flexible evaluation strategies depending on user intent and system confidence. Implicit models assess emotion and behavior in real-time, while explicit modules structure formal questionnaires and scoring rubrics. Both outputs contribute to a unified user profile and are interpretable within the context of validated psychological constructs.

More specifically, the implicit analysis module 361 continuously monitors user interactions, such as natural language input, sentiment trajectory, response latency, and behavioral markers, using large language models and trained classifiers. These machine learning components are calibrated on psychological signal patterns and enable passive inference of emotional distress, mood volatility, or cognitive overload without requiring formal user prompts.

FIG. 11 illustrates an implicit symptom detection subsystem according to an embodiment of the present disclosure. The implicit symptom detection subsystem may include a mechanism that evaluates user expression signals against a symptom knowledge base using a scoring framework. The subsystem may accumulate scores for detected symptoms, compare the aggregate score against a threshold, and generate a risk notification if a potential mental health issue is inferred.

More specifically, the implicit symptom detection subsystem may be embedded within the overall vertical AI agent framework. As shown in FIG. 11, the implicit symptom detection may be triggered when a user expression signal 1101 is received from the user, which may contain subtle indicators of emotional or psychological issues.

The user expression is passed to the symptom evaluation engine 1102, which analyzes the inputted user expression across a predefined list of potential symptoms (e.g., sadness, fatigue, hopelessness). Each symptom is sequentially evaluated based on semantic, emotional, or contextual cues extracted from the inputted user expression. Parallelly or simultaneously, a symptom scoring initialization unit 1103 may initialize a scoring variable to zero. As the evaluation proceeds, an incremental scoring logic unit 1104 adds ‘1’ (+1) to the score each time a corresponding symptom is detected.

After all evaluations are completed, the total score (the value of the scoring variable) is computed by the symptom score aggregator 1105. The total score is then passed to the threshold evaluation module 1106, which determines whether the user's symptom intensity surpasses a predefined threshold.

If the total score exceeds the predefined threshold (e.g., indicating possible mild-to-moderate depressive symptoms), a risk notification generator 1107 produces an internal flag or user-facing warning. Simultaneously, relevant anonymized data may be stored in the knowledge archiving & signal logging database 1108 for long-term training, personalization, and performance tracking.

The implicit analysis module 361 may enable real-time, passive detection of latent risks, allowing the system to take precautionary action even when users do not explicitly disclose their mental health status. In parallel, the explicit assessment module 362 may deliver structured, clinically validated instruments, such as DSM-5-based diagnostic scales, PHQ-9, or GAD-7, through interactive workflows within the user interface. These assessments are modular and dynamically selected based on user behavior, self-reported symptoms, or agent-triggered concern thresholds.

FIG. 13 illustrates a flow diagram of a user-driven assessment initiation process according to the present disclosure. The user-driven assessment initiation process may provide a mechanism for the system to present a psychological or behavioral assessment opportunity and to initiate the corresponding process only upon affirmative user consent.

More specifically, as shown in FIG. 13, an assessment prompt module 1301 asks the user whether the user wishes to take a specific assessment. After the user responds, a user response handler 1302 captures the user input and passes the user input to the response decision node 1303. If the user agrees (Yes), the assessment launcher 1304 initiates the corresponding test. This interaction may ensure that assessments are launched only with explicit user consent, and may be used in coordination with prior screening modules.

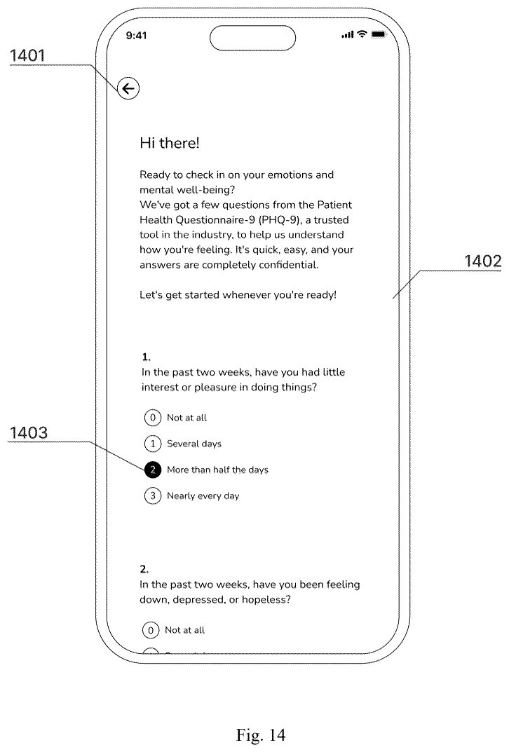

FIG. 14 shows the interactive diagnostic module interface according to an embodiment of the present disclosure. This user interface may deliver structured mental health assessments through dynamically generated question sets tailored to the user's psychological context.

As shown in FIG. 14, an example user interface is provided for administering a structured psychological assessment on a mobile device. The user interface includes a navigational element 1401 located near the top of the screen of the mobile device, allowing the user to return to the previous page. Below that, an instructional and contextual message area 1402 introduces the purpose of the assessment, such as screening for emotional well-being using tools like the PHQ-9. The central portion of the screen 1403 presents a multiple-choice format for individual assessment questions, enabling users to select from predefined response options. This layout is designed to support user engagement and confidentiality while facilitating structured data input for downstream processing.

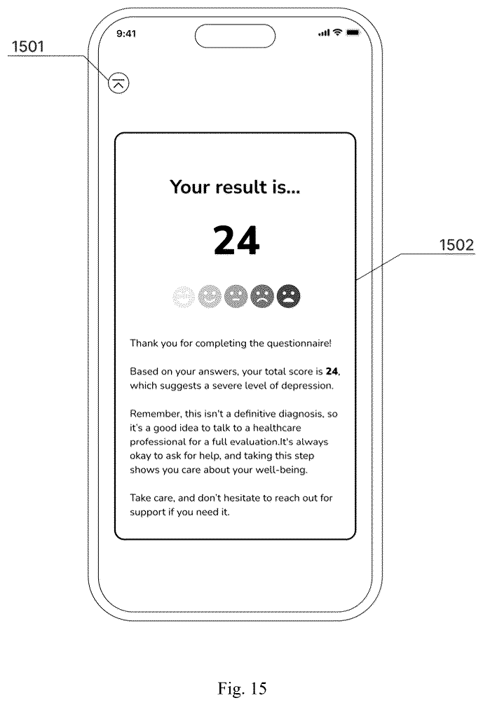

FIG. 15 illustrates a diagnostic results interface according to an embodiment of the present disclosure. The diagnostic results interface may display multi-dimensional scores, emotional indicators, and system-generated recommendations based on psychological evaluation algorithms.

As shown in FIG. 15, for example, a user interface is provided on a mobile device for displaying the result of a digital psychological assessment. The interface includes a navigational element 1501 allowing the user to exit or collapse the result view. The main display region 1502 prominently shows the user's computed score from the assessment, in this case, “24”, along with a corresponding emotional indicator using facial icons to convey severity. Below the score, explanatory text interprets the result, offering context and suggesting follow-up actions, such as seeking professional help. This layout ensures the user receives feedback that is both informative and empathetically delivered.

Further, a user's evolving state can be captured with the dynamic mental and psychological profile (MPP) engine 352 to create dynamic mental and psychological profile of the user. This dynamic profile tracks psychological indicators across dimensions such as emotional stability, self-acceptance, inner peace, and social connectedness. The MPP not only reflects the user's real-time mental health trajectory but also acts as a foundation for adaptive personalization across system modules.

The mental and psychological profile (MPP) is a structured, longitudinal container of user state. Each profile may include real-time emotional trends, history of prior diagnoses, preferred intervention modalities, and interaction cadence and behavioral tags, etc. The MPP may serve as a routing signal for content personalization, agent selection, and longitudinal progress tracking, and a visualization engines may optionally render the profile as radar charts, heat maps, or time-series graphs. The MPP for each user may be maintained continuously to create the core data structure for personalization, reasoning, and longitudinal monitoring.

The MPP also comprises multi-dimensional indicators reflecting the user's emotional, cognitive, and behavioral states. These dimensions may include, but are not limited to, emotional stability, self-acceptance, social connectedness, perceived life fulfillment, and capacity for rest or peace. Each dimension is populated through a combination of implicit inferences derived from natural language interaction, and explicit inputs from structured assessments administered through the system interface.

The MPP is dynamically updated in real time as the user interacts with the system. Updates occur through ingestion of diagnostic outputs from the hybrid diagnosis engine, behaviorally inferred emotional signals, and historical engagement patterns with recommendations or interventions. These data points are normalized, timestamped, and structured into a persistent and extensible profile schema.

In addition, the MPP may function as a foundational context layer for downstream personalization. It informs the selection, timing, and framing of recommendations delivered by the system's adaptive recommendation engine, and enables agents to generate more relevant, empathetic, and situationally appropriate responses. Additionally, it supports trajectory analysis, anomaly detection, and time-series visualization of psychological states, allowing both users and authorized professionals to observe progress and identify intervention needs.

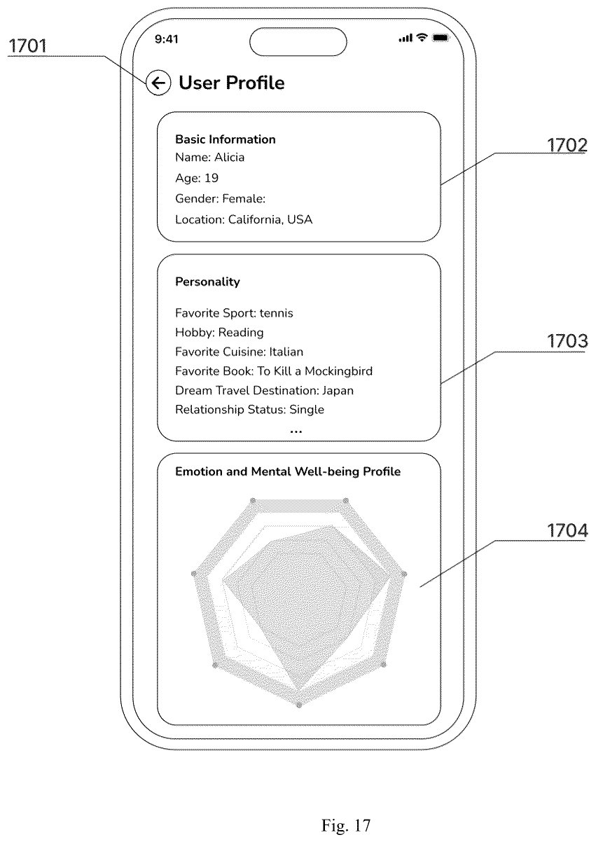

FIG. 17 illustrates a user interface of the MPP engine 362 according to an embodiment of the present disclosure. The user interface may be used to compile user-specific emotional and behavioral metrics into an interpretable format for downstream modules.

As shown in FIG. 17, for example, a graphical interface view may be generated by the MPP engine on a mobile device, integrating static user metadata with dynamic psychological state representation. The user interface may include a navigation element 1701, a metadata display unit 1702 rendering demographic and contextual attributes (e.g., age, gender, region), a personality insight component 1703 capturing lifestyle preferences, and a profile summary module 1704 showing an emotion and mental well-being profile. The profile summary module may include a visualized multi-axis distribution generated by the mental profiling engine, providing a compact representation of the user's inferred psychological baseline and current emotional profile.

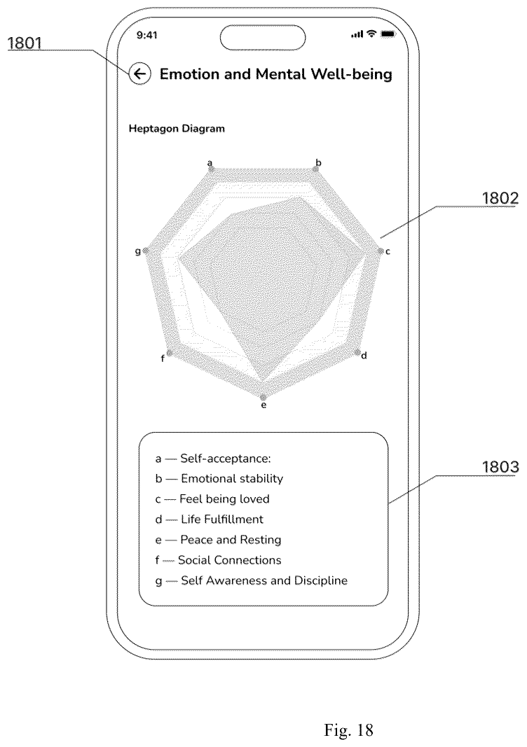

For example, the visualized multi-axis distribution may be displayed as a multilateral radar chart icon, each apex point may represent an axis (from the center of the icon to each apex point). Further, the user may click on the multilateral to show details of the multi-axis distribution. FIG. 18 illustrates a psychological factor decomposition view according to an embodiment of the present disclosure. The psychological factor decomposition view disaggregates the MPP into key sub-dimensions such as emotional resilience, self-perception, and social connectedness.

Specifically, as shown in FIG. 18, a navigation element 1801 may provide page navigation, and a heptagon diagram rendering module 1802 within the MPP system may visualize the distribution of psychological signal strength across seven predefined latent dimensions. These dimensions, mapped in the corresponding axis mapping layer 1803, may include: self-acceptance, emotional stability, perceived social support, life fulfillment, restfulness, social connectedness, and self-regulation. A score is calculated for each dimension and the apex points of the shaded shape represent the scores of all the dimensions. Each score is computed based on a combination of real-time self-assessment inputs and long-term behavioral signal aggregation, enabling system-level adaptive profiling for downstream applications such as personalized support generation or longitudinal mental health tracking.

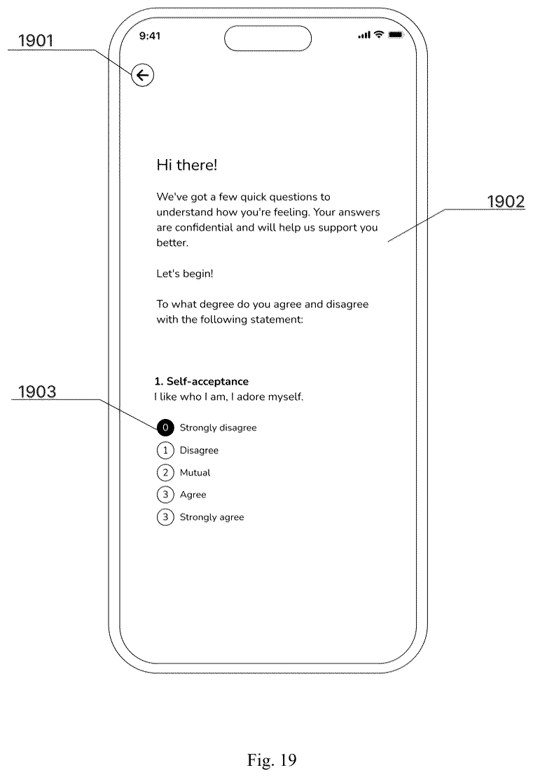

FIG. 19 shows an adaptive questionnaire user interface according to an embodiment of the present disclosure. The adaptive questionnaire user interface may be used to collect input signals relevant for updating and refining the user's MPP based on feedback loops and state changes.

Specifically, as shown in FIG. 19, a self-assessment signal acquisition interface is provided for a mobile device. The acquisition interface presents user-facing question items used to quantify latent psychological constructs. The interface includes a navigation element 1901, a prompt display module 1902, and a structured response selector (1903), supporting Likert-type input to assess agreement with targeted psychological statements. This input is continuously processed by the mental profiling engine to update the corresponding axis score, contributing to the system's temporal modeling of the user's mental state. This mechanism allows for iterative refinement of individual psychological profiles and supports multi-dimensional calibration for emotionally adaptive digital interventions.

Returning to FIG. 3B, to ensure precision and scalability in recommendation delivery, the system also includes the adaptive recommendation engine 353. The recommendation engine 353 operates across content categories, such as apps, videos, interventions, and live events, ranking them based on multi-signal scoring: (1) content-user relevance: based on tf-idf-like similarity across user clusters with shared conditions; (2) engagement history: prior success metrics in similar user segments; (3) expert tagging: hand-labeled scores from clinical experts or ai moderators; and (4) user feedback loops: incorporation of thumbs-up/down and behavioral drop-offs into real-time ranking logic. As such, the recommendation engine 353 allows the system to adapt continuously, refining its intervention strategy over time in alignment with the user's evolving mental state. Further, the recommendation engine 353 can tailor interventions, such as therapeutic exercises, lifestyle nudges, or multimedia content, by interpreting the user's MPP in conjunction with historical behavior and engagement signals. Ranking strategies include profile similarity, relevance scoring, and social graph-based optimization, etc.

The adaptive recommendation engine may operate based on relevance scoring, filtering, and collaborative enhancement. To support precision and scalability, the system may implement an intelligent content aggregation and filtering engine (standalone or integrated in the adaptive recommendation engine 353) that governs recommendation logic through multi-layered scoring and ranking mechanisms. Each content item is assigned a composite relevance score derived from: (1) semantic similarity between the item's metadata and the user's current MPP vectors (e.g., cosine distance or transformer-based embeddings), (2) empirical behavioral signals including usage frequency, dwell time, and subjective effectiveness ratings from matched user cohorts, and (3) expert-defined heuristic weights attached to clinical categories or intervention types.

In certain embodiments, a frequency-inverse-behavioral-impact scheme is applied, analogous to tf-idf, where term frequency is reinterpreted as observed effectiveness among similar psychological profiles. Filtering logic excludes low-signal or redundant items through automated and optionally human-in-the-loop review. Furthermore, the system maintains a similarity graph of user clusters derived from engagement trajectories and emotional feedback. When recommending content for a particular user, the engine consults this network to elevate content that has demonstrated positive outcomes in analogous psychological states. This hybrid strategy, merging vectorized matching, behavioral analytics, and profile-specific heuristics, enables adaptive, data-driven personalization at scale.

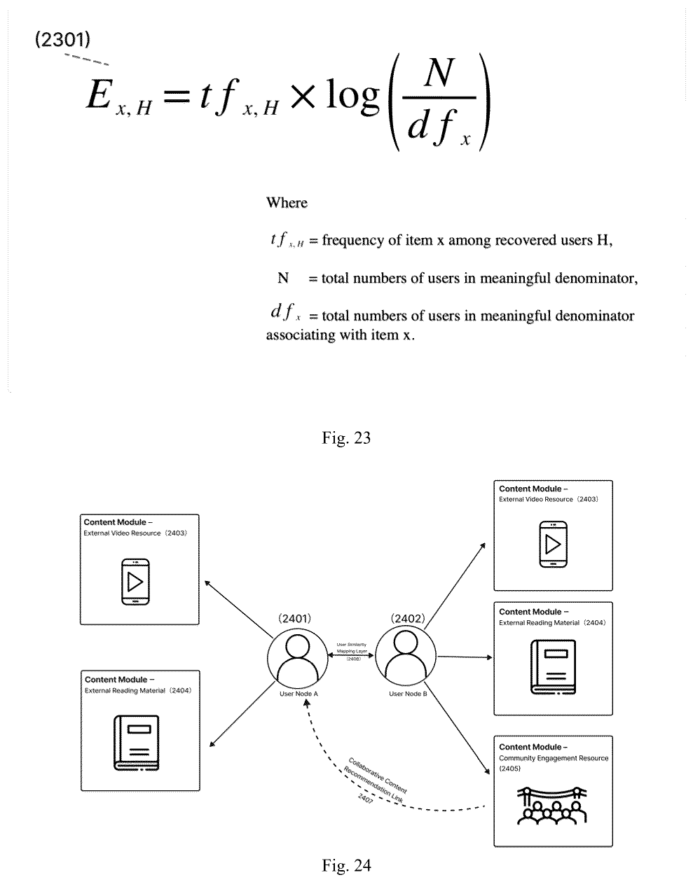

FIG. 23 illustrates the computational formula used to calculate content relevance scores, integrating recovery likelihood, user affinity, and topic alignment factors. As shown in FIG. 23, a tf-idf-inspired scoring model (2301) adapted to behavioral signals. The weight of each content item is calculated based on how frequently it appears among recovered users, normalized against its general prevalence.

In the formula

$${E}_{x , H} = {tf}_{x , H} \times log ( \frac{N}{{df}_{x}} )$$

tf_{x,H}=frequency of item x among recovered users H (e.g. among users who recovered from certain mental issue, what percentage of them used this item),
N=total numbers of users in meaningful denominator (e.g. total users, or total users who were ever diagonalized with certain mental health issues),
dfₓ=total numbers of users in meaningful denominator used item x.

Using this formula, the effective item to certain mental health issues will get a high score, the generally good items will get a medium score, and ineffective items will get a low score. This prioritizes content that is both effective and specialized.

Returning to FIG. 3B, the adaptive recommendation engine 353 may dynamically translate a user's evolving MPP into personalized therapeutic interventions. The adaptive recommendation engine 353 functions as a core downstream module, responsible for selecting, scheduling, and delivering targeted support materials including but not limited to digital therapeutics, psychoeducational resources, guided activities, and emotion-regulation tools. Recommendations can be triggered through various mechanisms such as scheduled intervals, real-time context detection, or task completion events. All outputs are logged and feedback-enriched to support continuous optimization. The architecture of the adaptive recommendation engine 353 may be modular and interoperable with third-party content repositories, ensuring broad compatibility across ecosystems while retaining personalization fidelity.

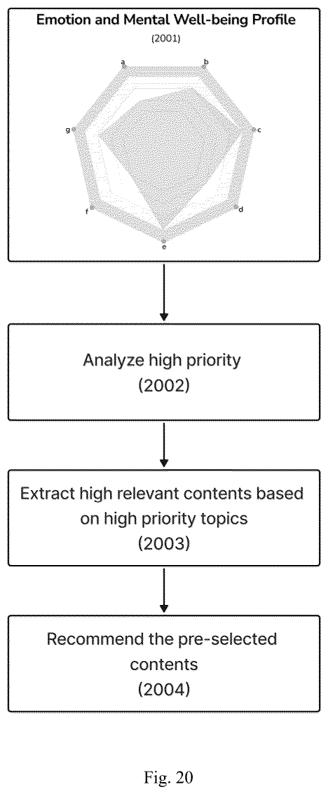

FIG. 20 illustrates a personalized recommendation process according to an embodiment of the present disclosure. The personalized recommendation process or pipeline transforms MPP vectors into tailored content suggestions via relevance inference and agent filtering.

Specifically, as shown in FIG. 20, the content recommendation process starts from the emotion and mental and psychological profile 2001, the system first identifies dimensions requiring focused support (i.e., analyze high priority) (2002), extracts high-relevance content based on high priority topics or aligned with those dimensions (2003), and generates a ranked recommendation list and recommends the pre-selected contents (2004). This diagram reflects the concept of translating user states into targeted support through structured prioritization and recommendation delivery.

To ensure psychological relevance and individualized support, the adaptive recommendation engine 353 may utilizes a profile-dimension-driven content matching strategy. Each support resource in the system is pre-tagged with structured metadata capturing its emotional tone, cognitive demand, intervention type, therapeutic intent, and intended target population. These metadata vectors are algorithmically aligned with the user's MPP vectors, representing dimensions such as emotional stability, self-acceptance, life fulfillment, social connection, and peace of mind. When gaps or low-scoring regions are detected within the MPP, the engine elevates content mapped to those specific axes, thereby directing user attention to high-need domains. Such configuration may enable the system to proactively surface content that addresses the most salient or underdeveloped aspects of a user's psychological state.

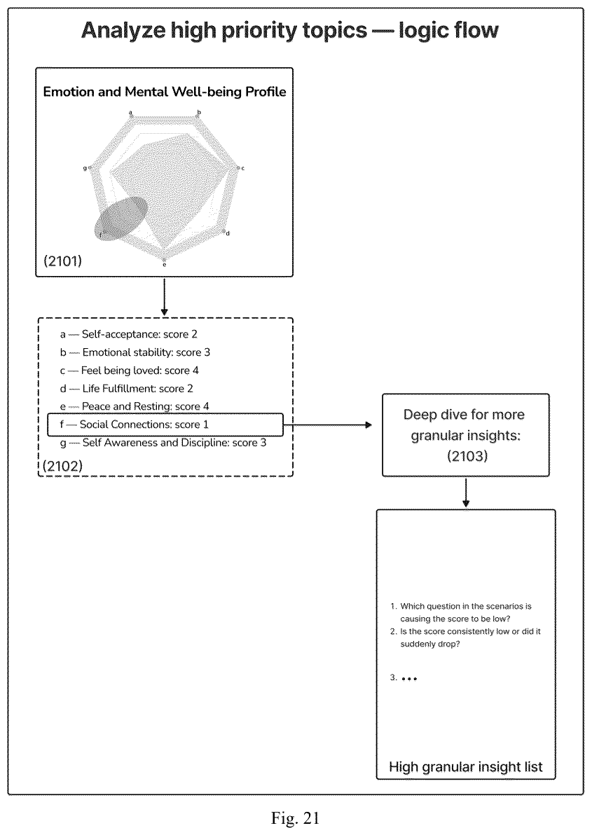

FIG. 21 illustrates a flow diagram of a process for analyzing high priority topics according to an embodiment of the present disclosure. The process may be used for prioritizing psychological focus areas, using signal weighting, user profiling, and historical response modeling.

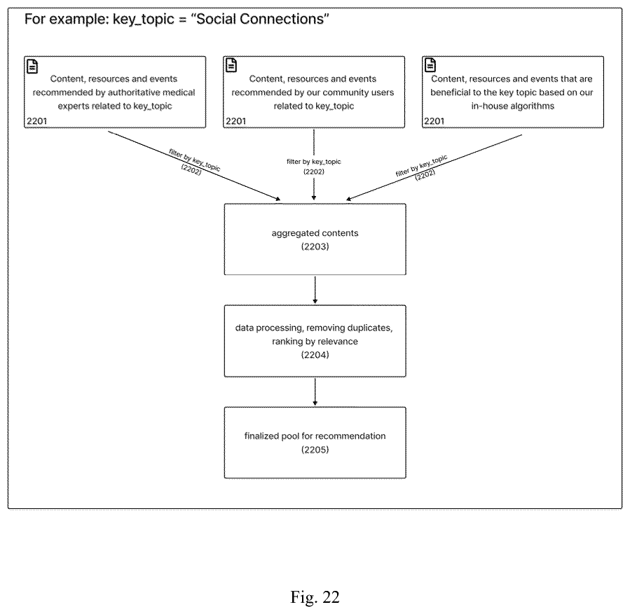

FIG. 22 illustrates a flow diagram of particular dimension processing according to an embodiment of the present disclosure. A resource extraction module may be used to rank and retrieve relevant content items aligned with a high-priority psychological domain.

FIGS. 21 and 22 demonstrate how the system performs personalized dimension-driven analysis. From the user's radar chart profile (2101), the system parses scores for each emotional or psychological attribute in each dimension (2102). Key low-performing dimensions (e.g., social connections with score 1) are flagged for deeper inspection. The system then triggers a granular breakdown (2103), identifying contributing questions, score trends, or sudden shifts (2104). For example, when key_topic is “social connection”, various content, resources, and events recommended by authoritative medical experts related to key_topic 2201 are filtered by key_topic (2202) to form aggregated contents 2203. The aggregated contents are processed (e.g., data processing, removing duplicates, ranking by relevance, etc.) (2204) to form finalized pool for recommendation (2205). These insights support targeted matching between user-specific psychological needs and pre-indexed content categories.

FIG. 24 illustrates a collaborative filtering framework according to an embodiment of the present disclosure. The collaborative filtering framework provides a collaborative filtering mechanism for mental health content, utilizing a similarity network across anonymized user profiles to enhance personalization.

Specifically, as shown in FIG. 24, a similarity engine or a user similarity mapping layer 2408 may identify psychologically similar users (2401, 2402). If any content such as external videos (2403), books (2404), or local communities (2405) was effective for one user, the system can recommend it to another similar user (2407). This behavioral reinforcement loop ensures relevance beyond static metadata, enhancing the personalization engine with live community-driven intelligence.

Accordingly, to enable precise and adaptive delivery of personalized support content, the intelligent content aggregation and filtering engine may interface directly with the user's dynamically evolving MPP. Upon each profile update, the system identifies high-priority psychological dimensions that exhibit stagnation, sharp decline, or persistently low scores. These dimensions serve as focal points for downstream matching and intervention. This prioritization workflow is illustrated in FIG. 20-21 as an example, but implementations may accommodate additional or alternative prioritization logic.

The relevance of each candidate content item is scored through a multi-factor algorithm. This score reflects both semantic alignment between the item and the user's profile vector, and behavioral indicators such as prior effectiveness within similar psychological cohorts. Time-sensitive context, interaction history, and domain-specific weightings are optionally incorporated. In certain embodiments, a tf-idf like formula is used to emphasize items that demonstrate higher success rates among recovered or high-outcome users, as exemplified in FIG. 23. This formulation is illustrative only and does not limit the system to a particular scoring equation.

Content aggregation is executed continuously from multiple input streams, including domain-expert repositories, user-submitted materials, and in-house algorithmic generators. All items are filtered based on indexed dimensions, passed through a deduplication and relevance-checking layer, and ranked accordingly. FIG. 22 presents one possible flow of this pipeline, showing how content from diverse sources is tagged, filtered, and selected, but this structure is modular and extensible beyond the specific inputs shown.

To further enhance personalization, the system may incorporate a collaborative layer that leverages user similarity networks. When users exhibit matching psychological patterns or engagement histories, successful content consumed by one user can be propagated to others with analogous needs. This process is illustrated in FIG. 24, where collaborative links are formed between similar user nodes to transmit content modules (e.g., video, reading, community tools). These modules serve only as examples; the system is not limited to these categories and may include any content type relevant to the psychological context.

Similarity among users may be identified based on their historical engagement with contents, products on the platform. After the users' similarity network is established, when a user faces certain mental issues, what item is useful may be identified among this user's similar network for the same kind of issue, and such item may be recommended to the user. For example, a user experiencing symptoms of anxiety and depression may log into the system using secure multi-factor authentication, and interact with a chatbot to express the feelings of anxiety. The chatbot or other UI component may utilize an anxiety management agent to provide the user with mindfulness exercises and breathing techniques. Over time, the user's interactions are analyzed, and the system detects persistent signs of depression. The chatbot may then guide Jane through a structured diagnostic assessment based on DSM-5 criteria. The results are stored in the user's MPP, based on which the system provides personalized recommendations, including lifestyle adjustments, content recommendations like applications and videos, and a referral to a professional therapist, etc.

Returning to FIG. 3A, To ensure secure and controlled access to mental health services, the system may use a user authentication and interface layer (e.g., as implemented by authentication and access control 305 and/or user device 302) featuring various authentication mechanisms, such as multi-factor verification, session token management, and user role mapping. This mechanism enforces tiered access permissions and protects sensitive health data in accordance with system-level privacy policies and user-defined consent rules.

Once authenticated, the user may interact with a multi-component interface (i.e., user interface or UI) designed specifically for emotional and psychological contexts. The front-end layer supports both mobile and web deployment and includes: (1) a real-time chat interface for dialog with orchestrated AI agents; (2) a resource library that provides access to multimedia mental health content; and (3) a user dashboard where individuals can review diagnostic results, emotional trends, and recommendation history.

The interface may adapt to each user's MPP, ensuring that prompts, content, and tone are aligned with the user's current state. Certain design considerations include emotional safety, clarity of navigation, and visual responsiveness across device types.

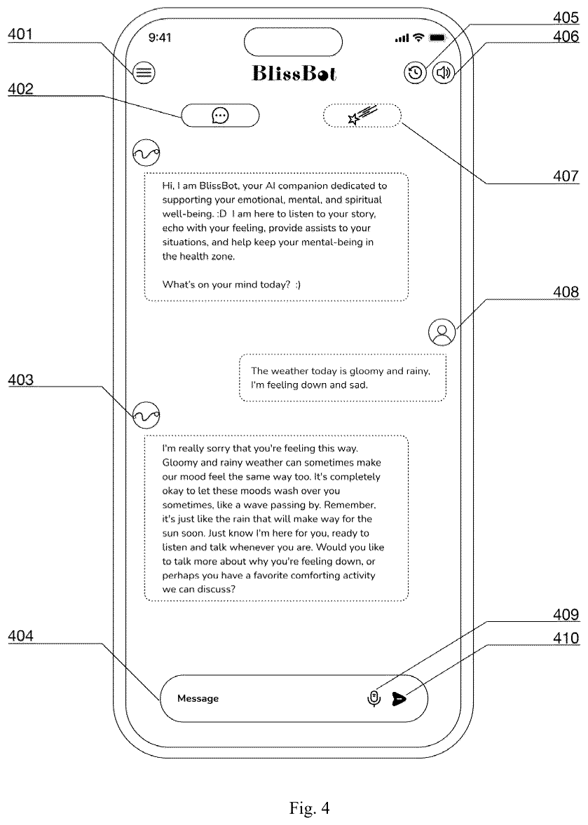

FIG. 4 illustrates a user interface of the system (named BlissBot) designed for AI-based mental and emotional support according to an embodiment of the present disclosure. The interface includes a status bar (not labelled) at the top of the user device 401 indicating device and time information; a navigation menu icon 401 opening the main menu or navigation options for different app sections; and a chat mode toggle 402 switching to the chat interface where users interact with the AI agent via text. The interface also includes a chatbot avatar 403, a visual icon representing the ai assistant in the chat, enhancing user engagement; a text input field 404, an area where the user types messages to communicate with the ai; and historical chat information 405 displaying access or indicator for past chat conversations and session history. The interface also includes system notifications 406 showing alerts or system messages related to app status or user interactions; and a switch to “make a wish” feature 407, which may be a button to toggle to the “make a wish” module for wishful or positive affirmations. The interface also includes a user avatar 408, a visual icon representing the user, typically used to identify user messages.; a voice message button 409 enabling recording and sending voice messages to the ai assistant; and a send message button 410 sending the current typed or recorded message to the ai for processing.

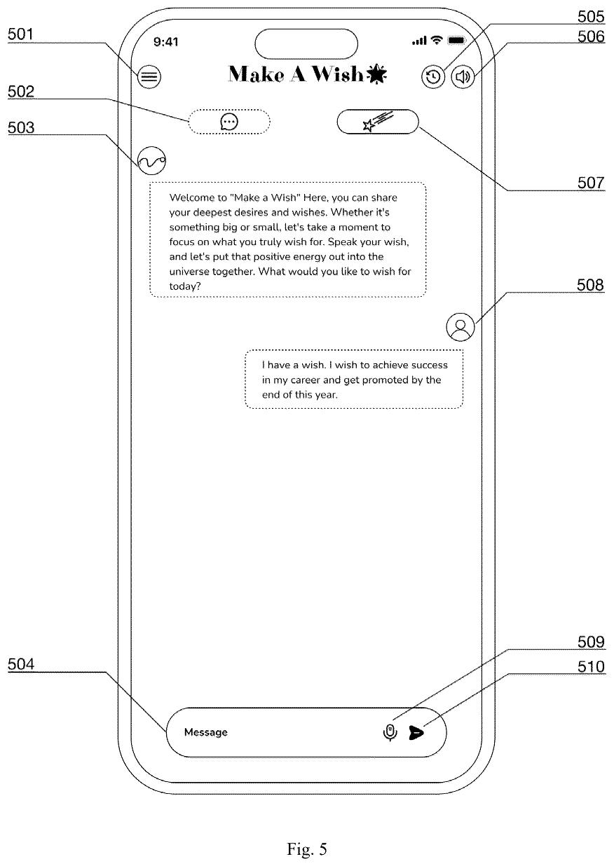

FIG. 5 illustrates another user interface architecture within the BlissBot system according to an embodiment of the present disclosure. The interface includes navigation menu icon 501 opening the main menu or navigation options for different app sections; a chat mode toggle 502 switching to the chat interface where users interact with the AI agent via text; and a chatbot avatar 503, which may be a visual icon representing the ai assistant in the chat, enhancing user engagement. The interface also includes a text input field 504, an area where the user types messages to communicate with the AI; historical chat information 505 displaying access or indicator for past chat conversations and session history; and system notifications 506 showing alerts or system messages related to app status or user interactions. The interface also includes a switch to chat mode button 507, toggling the interface back to the main chat or conversational mode from the “make a wish” feature; a user avatar 508, a visual icon representing the user, typically used to identify user messages; a voice message button 509 enabling recording and sending voice messages to the ai assistant; and a send message button 510 sending the current typed or recorded message to the ai for processing. This interface supports structured emotional disclosure aligned with user intent and system response architecture.

These UI components collectively provide a secure, accessible, and therapeutically optimized user experience. They serve not only as input/output channels, but also as dynamic containers for monitoring user progress, managing privacy settings, and engaging with personalized interventions.

Returning to FIG. 3B, to address the heightened sensitivity of mental health data, the system also includes the privacy control and user-defined data control engine 354 as the privacy control infrastructure and user-defined data governance, which enables users to select their preferred data handling policy. This configuration allows the system to support diverse user expectations regarding confidentiality, intervention, and research participation, while ensuring regulatory compliance.

More specifically, upon onboarding, or at any time via account settings, each user may select one of the following data-sharing policies:

### Mode A: No Retention (Strict Privacy)

In this mode, no health-related information is stored or retained. All interaction data is processed in-memory and deleted upon session termination. Features such as longitudinal tracking, history-based MPP updates, or continuity recommendations are disabled.

### Mode B: Private Use Only (Personalized Without Sharing)

The system stores health-related interaction data for internal personalization, including dynamic MPP generation, implicit analysis, and continuity of care. However, no information is shared with third parties or accessible by internal experts beyond the user-facing system logic.

### Mode C: Internal Expert Support Enabled

Users may authorize the system to share their data with in-house medical staff, licensed professionals, or designated expert AI agents. These experts may analyze user data and intervene when clinically significant patterns emerge. All such access is subject to strict role-based control and audit logs.

### Mode D: Research and ai Training Contribution

Users may voluntarily contribute anonymized data for use in population-level mental health research, public health modeling, or large-scale language model training. Only de-identified data is shared, and users are notified about its intended use. Opt-out rights are preserved at all times.

These policies govern data flow across all modules—including hybrid diagnostics, MPP construction, recommendation engines, agent decision logs, and system analytics. User preferences are cryptographically recorded and retroactively enforced where technically feasible. Consent may be modified or revoked by the user at any time through a unified privacy interface.

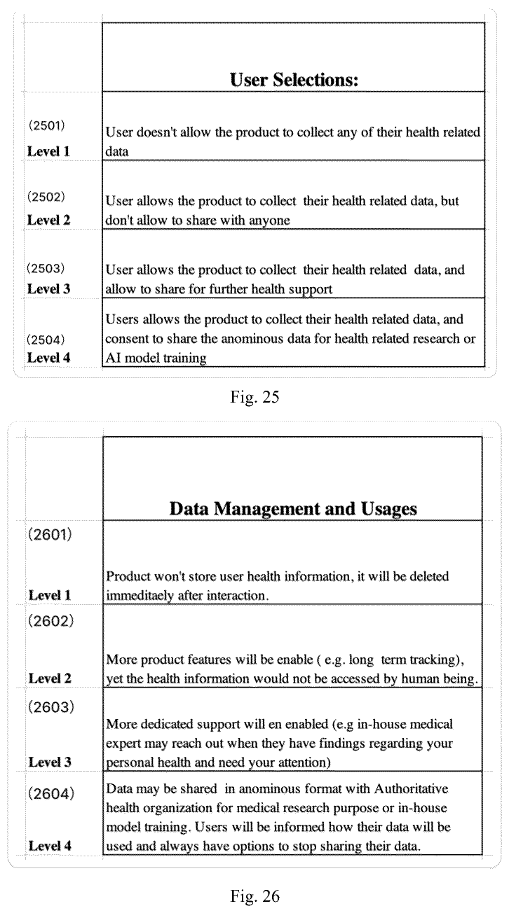

FIG. 25 illustrates a privacy setting interface according to an embodiment of the present disclosure. The privacy setting interface may enable a user to manage data-sharing preferences, consent boundaries, and visibility scopes of sensitive information.

Specifically, as shown in FIG. 25, a configurable interface is provided and, through the interface, the user may specify certain preferences regarding data collection and usage. The system may supports multiple levels of consent. At Level 1 (2501), the user declines all health-related data collection, and the system is limited to generic functionalities. At Level 2 (2502), the user permits data collection for internal personalization but disallows any sharing. At Level 3 (2503), data may be shared with authorized third parties for the purpose of improving individual support. At Level 4 (2504), the user consents to anonymized data being used for population-level research or AI model training. This consent framework enables dynamic adjustment of system capabilities while maintaining user autonomy and regulatory compliance.

FIG. 26 illustrates a system-level architecture diagram of data management and usage according to an embodiment of the present disclosure. The data management architecture may be used for controlling secure storage, masked identity mapping, and encrypted data access pathways.

Specifically, as shown FIG. 26, a tiered data management structure may be used (Levels 2601-2604), where each level defines a distinct boundary of data retention and usage. At Level 1 (2601), no user health data is retained, and interactions are deleted immediately. Level 2 (2602) enables expanded features such as long-term tracking, while ensuring data remains machine-accessible only. Level 3 (2603) allows internal experts to review data and initiate support when needed. At Level 4 (2604), a user authorizes anonymized data to be shared for research or model training, with full transparency and opt-out control. This structure supports flexible compliance and user-centric governance across varying privacy expectations.

In parallel with user-facing privacy controls, the system provides tiered access control and confidential data mapping that separates health-related information by sensitivity level, access purpose, and functional role. The confidential data mapping may be based on a masked identifier (MID) protocol that abstracts user identity from operational and analytical workflows.

Each user is assigned a MID that is used throughout all data processing and storage modules. Personally identifiable information (PII)—including name, email, and demographic markers—is stored separately and mapped to the MID through a secured and encrypted identity vault.

Access to different categories of data may be controlled as follows:

### Confidential Level 1: Identity Mapping Layer (Highest Confidentiality)

The MID-to-real-identity mapping is accessible only to a restricted group of system operators—such as lead data engineers, healthcare leads, or emergency escalation responders. Access requires dual authentication, written justification, and approval from a privacy governance committee. Access to this layer is limited to rare, critical use cases (e.g., suicide risk escalation or legal compliance requests).

### Confidential Level 2: Anonymized Health Data Layer (Very High Confidentiality)

A select group of internal personnel—typically comprising less than 10% of employees—may access anonymized user data tagged with MIDs for system development, AI model validation, or internal research. These roles may include core developers, data scientists, and credentialed mental health professionals. Access is granted only on a per-project basis, subject to committee approval and real-time usage logging.

### Confidential Level 3: Aggregated Health Metrics Layer (High Confidentiality)

Teams involved in population analytics, product optimization, or cohort-based trend detection may access data at an aggregated level (e.g., average scores, distributional changes). Access to this layer is restricted to anonymized groups with more than 20 individuals per cohort to maintain statistical privacy (k-anonymity). This layer does not support user reidentification.

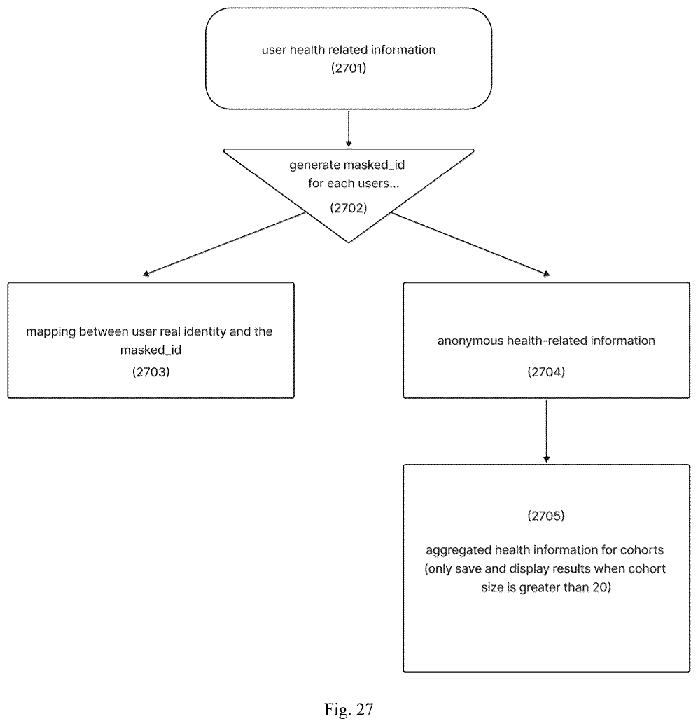

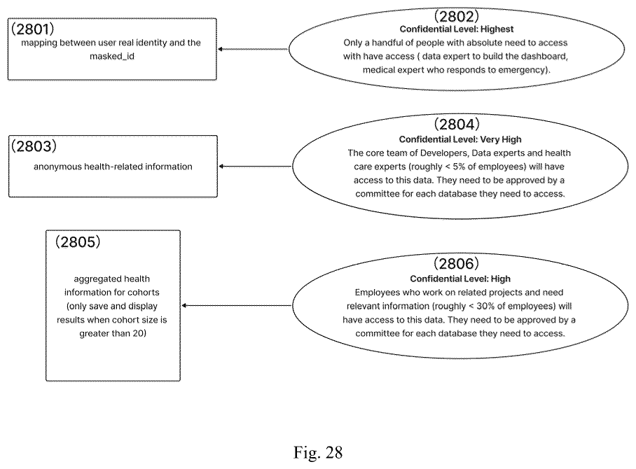

FIG. 27 illustrates a process for privacy-preserving data according to an embodiment of the present disclosure. The privacy-preserving data process may incorporate compliance enforcement, multi-level access control, and audit tracking.

Specifically, as shown in FIG. 27, a privacy-preserving data transformation process is provided for handling user health information. Upon intake of user health related information (2701), the system may generate a masked identifier (masked_id) for each user (2702), separating identity from data. The mapping between masked_id and real identity (2703) is stored separately under more strict controls, while the masked data is retained anonymously as anonymous health-related information (2704). Only de-identified information is used for further analysis. Further, aggregated cohort-level outputs (2705) are generated only when a minimum number of users (e.g., >20) is met, reducing re-identification risk.

FIG. 28 illustrates an internal role-based access control (RBAC) framework according to an embodiment of the present disclosure. The RBAC framework may be designed to ensure only authorized personnel or modules can retrieve identifiable mental health records.

As shown in FIG. 28, a corresponding access control framework is provided based on data sensitivity. The identity mapping (2801) is restricted to being accessed by essential personnel with highest clearance or under “highest” confidentiality level (2802). Anonymous health records (2803) are accessible only to a core data and clinical team under “very high” confidentiality (2804). Aggregated insights (2805) are made available to approved staff working on relevant projects under “high” confidentiality (2806). All access is committee-reviewed and logged to ensure compliance.

In addition, all data access activity, across levels, may be cryptographically logged, timestamped, and periodically reviewed through an internal audit console. The access control system may be integrated with the user-facing privacy preferences, ensuring that no data access, regardless of level, violates the user's selected governance mode.

Users may be informed via automated notifications whenever elevated access to their anonymized data is requested (e.g., for research participation). The users can review access logs and revoke data processing permissions at any time, under full compliance with data protection laws such as HIPAA, GDPR, and related global standards. Accordingly, the privacy control and user-defined data control engine 354 may provide privacy-aware storage and retrieval, with components decoupled into identifiable dimensions, each governed by user-defined sharing preferences and role-based access rules.

During operation, the system can use the various subsystems/modules described above to implement various processes such as user interaction, diagnostic, data management, personal recommendation, etc. Although these processes are not described explicitly in the entirety, those skilled in the art can implement those processes by combining functionalities of the various subsystem and other described processes.

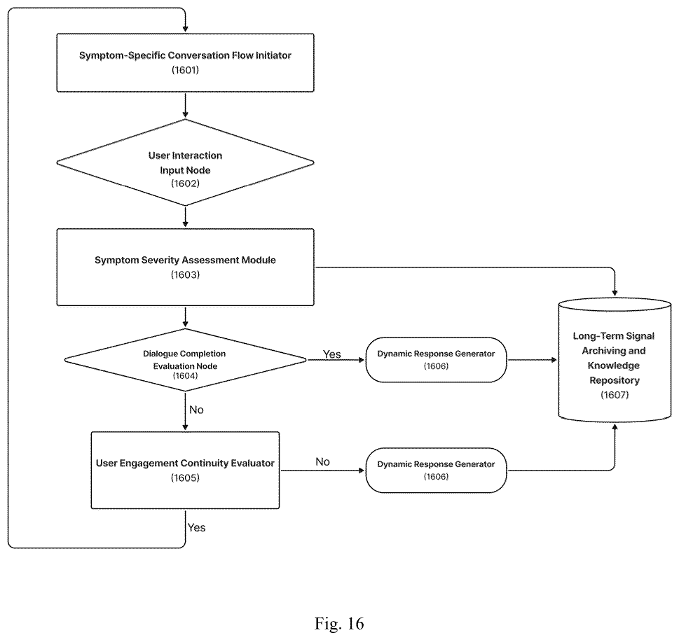

FIG. 16 illustrates a flow diagram of an adaptive interaction process according to an embodiment of the present disclosure. The adaptive interaction process may be provided for assessing and responding to user symptom-related signals, in which conversation modules dynamically assess severity, evaluate dialogue completion, and determine continuity of engagement, with relevant data archived in a long-term signal repository.

Specifically, as shown in FIG. 16, at the beginning of a dynamic dialogue flow driven by symptom-specific context, a symptom-specific conversation flow initiator 1601 starts the interaction, and a user input is captured via the user interaction input node 1602. The symptom severity assessment module 1603 analyzes the user input and evaluates symptom intensity. Further, a dialogue completion evaluation node 1604 checks whether the dialogue task is completed. If the dialogue task is completed (Yes), a dynamic response generator 1606 produces a closing response and archives the interaction in the long-term signal archiving and knowledge repository 1607. If the dialogue task is not completed (No), the user engagement continuity evaluator 1605 determines if the user is still engaged. Based on the outcome of the determination, the system flow either continues the conversation or concludes it accordingly.

During operation of the AI-based system, the various frameworks, AI agents, and other components described herein may be configured in various ways to support online mental health services. To illustrate an application of the disclosed system, an example of a typical use case in which a user exhibits early signs of psychological distress and receives end-to-end support through the integrated platform of the AI-based system.

From any user device, a user may log into the system through the secure authentication module and begin interacting with the AI interface (e.g., UI, chatbot, etc.). During the initial conversation, the implicit analysis module may detect indicators of elevated anxiety, such as fast-paced input on the user device, negative sentiment trajectory, and linguistic markers associated with distress. The system may trigger the hybrid diagnosis engine to combine this implicit signal with an explicit assessment, such as the GAD-7 anxiety screening form, presented through the interface.

The results from both modules may then be aggregated into a contextualized state vector and written to the user's mental and psychological profile (MPP). Based on this update, the system identifies multiple low-sufficiency dimensions, specifically emotional stability and peace/rest, and prioritizes them for immediate intervention.

Further, the adaptive recommendation engine retrieves a ranked list of support content using a blended scoring model. High-ranking items include guided breathing exercises, a 5-minute grounding video, and a digital journaling activity. These recommendations are drawn from content previously found effective among users with similar profiles (via collaborative filtering), and validated by semantic relevance to the MPP vector.

Over the following days, the user continues engaging with the chatbot. The system observes a reduction in anxiety signal strength, updates the MPP accordingly, and shifts its recommendation strategy toward long-term lifestyle alignment. All data processing and personalization logic operate within the system's tiered privacy infrastructure, and the user retains control over their data visibility and consent settings.

The above example demonstrates the real-time coordination of multi-agent reasoning, semantic knowledge retrieval, hybrid diagnostics, personalized profiling, and adaptive content delivery as disclosed in the present invention.

Accordingly, the disclosed system, processes, and components may represent a significant advancement in AI-based mental health support, addressing the high costs, limited accessibility, and privacy concerns associated with traditional methods. By leveraging LLMs, RAG agents, a mixed expert agent collaboration model, and other frameworks and components, the system provides comprehensive, empathetic, and contextually relevant mental health diagnosis, treatment, and support, ensuring that users receive precise and timely mental health care, ultimately improving overall well-being.

Accordingly, the present disclosure provides a system architecture that provides scalable, adaptive, and personalized support through the integration of large language models (LLMs) and a vertical AI agent framework. The system includes multiple specialized agents, each assigned to a domain-specific function, collaborating to analyze user input, generate responses, and support cognitive and emotional tasks in real time. A semantic knowledge engine, such as a vector-based or equivalent database, is employed to embed structured and unstructured domain literature into machine-readable representations, enabling contextual retrieval and semantic matching. The system further incorporates both implicit and explicit diagnostic engines to interpret user states from conversational input and validated assessments. A dynamically updated psychological profiling module captures users' mental, emotional, and spiritual dimensions, which in turn informs a recommendation engine capable of delivering content and interventions personalized to the individual. The present disclosure ensures data security through tiered privacy infrastructure and is extensible beyond mental health to educational, medical, and other industrial applications requiring domain-specific intelligent support

That is, the present disclosure also provides a modular and extensible system architecture for delivering scalable, adaptive, and contextually relevant mental health support through AI-driven mechanisms. The disclosure integrates a vertical AI agent framework, wherein a leader orchestration agent coordinates a set of domain-specific expert agents, each configured to interpret user input and provide specialized support across distinct psychological functions.

The system utilizes large language models (LLMs) enhanced through prompt engineering and, in certain embodiments, domain-specific fine-tuning or self-training using therapeutic corpora. These agents operate over a shared semantic knowledge space constructed via a knowledge embedding and semantic retrieval engine, which transforms structured and unstructured psychological literature into vectorized representations for real-time contextual reasoning.

Each user is continuously represented by a personalized mental and psychological profile (MPP), which captures dynamic attributes across multiple mental, emotional, and behavioral dimensions. This MPP serves as the basis for all system personalization, driving the operation of an adaptive recommendation engine that selects and ranks support content and interventions matched to the user's evolving state.

To ensure ethical and compliant deployment, the system includes a tiered privacy infrastructure with support for masked identity protocols, configurable consent layers, and multi-level access control. Users are given granular control over their data retention and sharing preferences, and internal access to sensitive data is limited to designated roles under formal authorization processes.

The disclosure is also suitable for implementation across various domains, including consumer-facing wellness platforms, enterprise well-being programs, educational environments, and clinical decision support systems. Its multi-layer design allows for seamless integration with evolving diagnostic frameworks, content repositories, or advancements in generative AI and emotional modeling.

In the descriptions of this specification, descriptions using reference terms “an embodiment”, “some embodiments”, “an exemplary embodiment”, “an example”, “a specific example”, or “some examples” mean that specific characteristics, structures, materials, or features described with reference to the embodiment or example are included in at least one embodiment or example of the present disclosure. In this specification, schematic descriptions of the foregoing terms are not necessarily directed at a same embodiment or example.

Further, in the descriptions of this specification, although certain parts or components are labeled with different reference numbers, those parts or components may be the same or similar, as long as they perform same or similar functions or have same or similar characteristics. Further, although various frameworks, infrastructures, agents, and other software components are described separately, they may be combined in any appropriate ways (even not explicitly described herein) by those skilled in the art.

Although embodiments of the present disclosure have been shown and described, a person of ordinary skill in the art should understand that various changes, modifications, replacements and variations may be made to the embodiments without departing from the principles and spirit of this application, and the scope of the present disclosure is as defined by the appended claims and their equivalents.

What is claimed is:

1. A method for providing online mental health support using an artificial intelligence (AI) based system including an orchestration AI agent and a plurality of vertical expert AI agents in a vertical expert AI agent pool, each expert AI agent being designed for a specific domain capability, the method comprising:
obtaining user input information from a user interface of a user device inputted by a user;
processing the user input information to generate a list of subtasks for responding to the user input information;
based on relevance scores of the plurality of vertical expert AI agents, assigning the list of subtasks to the plurality of vertical expert AI agents with each subtask assigned a most suitable vertical expert AI agent, wherein a relevance score of a vertical expert AI agent reflects a degree of relevance between a subtask and the specific domain capability of the vertical expert AI agent;
collecting responses from the plurality of vertical expert AI agents; and
synthesizing the responses from the plurality of vertical expert AI agents to create a final message personalized to the user input information, wherein the user input information includes both explicit information and implicit information.

2. The method according to claim 1, wherein the assigning the list of subtasks to the plurality of vertical expert AI agents with each subtask assigned a most suitable vertical expert AI agent further comprises:
for each subtask, computing relevance scores of the plurality of vertical expert AI agents with respect to the subtask, and assigning the subtask to one of the plurality of vertical expert AI agents with a highest relevance score as the most suitable vertical expert AI agent.

3. The method according to claim 1, further comprising:
storing the user input information, the list of subtasks, the responses, and the final message into a vector database; and
dynamically updating and training the plurality of vertical expert AI agents using the vector database.

4. The method according to claim 3, further comprising:
converting structured and unstructured domain content into high-dimensional vectorized representations using machine learning models; and
storing the high-dimensional vectorized representations into the vector database for contextual understanding and retrieval of information stored.

5. The method according to claim 1, wherein the user information includes natural language interactions, and the method further comprises:
processing, by an implicit analysis module, the natural language interactions using machine learning models to detect emotional, psychological, or behavioral signals;
performing, by an explicit assessment module, structured evaluations based on validated domain-specific criteria or clinical scales; and
combining outputs from both the implicit analysis module and the explicit assessment module to generate a contextualized diagnostic inference of a state of the user.

6. The method according to claim 5, further comprising:
dynamically updating a mental and psychological profile (MPP) of the user, which reflects the state of the user, based on ongoing interactions and diagnostic results; and
using the MPP to personalize support and recommendations to address specific mental health needs of the user.

7. The method according to claim 6, wherein the using the MPP to personalize support and recommendations to address specific mental health needs of the user further comprises:
utilizing behavioral data, interaction patterns, emotional and contextual signals, and historical outcomes of users from multiple sources to determine content relevance of content items in a curated content pool;
selecting a plurality of content items from the content items in the curated content pools based on the content relevance and the MPP of the user as personalized content items; and
delivering the personalized content items to the user through the user interface as personalized content recommendations.

8. The method according to claim 7, wherein the content relevance of content items in a curated content pool is determined by:
$${E}_{x , H} = {tf}_{x , H} \times log ( \frac{N}{{df}_{x}} ) ,$$
wherein tf_{x,H} is frequency of a content item x among users H who recovered from a certain mental health issue, N is a total number of the users who were ever diagonalized with the certain mental health issue, and dfₓ is a total number of users used the content item x.

9. The method according to claim 1, wherein the plurality of vertical expert AI agents are trained using existing pre-trained large language models (LLMs) through a prompt engineering mechanism, including crafting specific prompts to guide the pre-trained LLMs in generating contextually relevant and supportive responses tailored to mental health scenarios.

10. The method according to claim 1, wherein the plurality of vertical expert AI agents are trained via self-training LLMs using a curated dataset of mental and psychological conversation data, including therapy session transcripts, mental health applications, and anonymized user data, through steps of data preprocessing, supervised learning, and fine-tuning to improve the self-training LLMs to understand and respond to mental health-related queries.

11. The method according to claim 1, wherein the user interface includes a chat window for real-time interaction, a resource library for accessing mental health content, and user profile management for updating personal information and tracking mental health progress.

12. A non-transitory computer-readable storage medium containing computer-executable instructions for, when executed by one or more processors, performing a method for providing online mental health support using an artificial intelligence (AI) based system including an orchestration AI agent and a plurality of vertical expert AI agents in a vertical expert AI agent pool, each expert AI agent being designed for a specific domain capability, the method comprising:
obtaining user input information from a user interface of a user device inputted by a user;
processing the user input information to generate a list of subtasks for responding to the user input information;
based on relevance scores of the plurality of vertical expert AI agents, assigning the list of subtasks to the plurality of vertical expert AI agents with each subtask assigned a most suitable vertical expert AI agent, wherein a relevance score of a vertical expert AI agent reflects a degree of relevance between a subtask and the specific domain capability of the vertical expert AI agent;
collecting responses from the plurality of vertical expert AI agents; and
synthesizing the responses from the plurality of vertical expert AI agents to create a final message personalized to the user input information, wherein the user input information includes both explicit information and implicit information.

13. The non-transitory computer-readable storage medium according to claim 12, wherein the assigning the list of subtasks to the plurality of vertical expert AI agents with each subtask assigned a most suitable vertical expert AI agent further comprises:
for each subtask, computing relevance scores of the plurality of vertical expert AI agents with respect to the subtask, and assigning the subtask to one of the plurality of vertical expert AI agents with a highest relevance score as the most suitable vertical expert AI agent.

14. The non-transitory computer-readable storage medium according to claim 12, further comprising:
storing the user input information, the list of subtasks, the responses, and the final message into a vector database; and
dynamically updating and training the plurality of vertical expert AI agents using the vector database.

15. The non-transitory computer-readable storage medium according to claim 14, further comprising:
converting structured and unstructured domain content into high-dimensional vectorized representations using machine learning models; and
storing the high-dimensional vectorized representations into the vector database for contextual understanding and retrieval of information stored.

16. The non-transitory computer-readable storage medium according to claim 12, wherein the user information includes natural language interactions, and the method further comprises:
processing, by an implicit analysis module, the natural language interactions using machine learning models to detect emotional, psychological, or behavioral signals;
performing, by an explicit assessment module, structured evaluations based on validated domain-specific criteria or clinical scales; and
combining outputs from both the implicit analysis module and the explicit assessment module to generate a contextualized diagnostic inference of a state of the user.

17. The non-transitory computer-readable storage medium according to claim 16, further comprising:
dynamically updating a mental and psychological profile (MPP) of the user, which reflects the state of the user, based on ongoing interactions and diagnostic results; and
using the MPP to personalize support and recommendations to address specific mental health needs of the user.

18. The non-transitory computer-readable storage medium according to claim 17, wherein the using the MPP to personalize support and recommendations to address specific mental health needs of the user further comprises:
utilizing behavioral data, interaction patterns, emotional and contextual signals, and historical outcomes of users from multiple sources to determine content relevance of content items in a curated content pool;
selecting a plurality of content items from the content items in the curated content pools based on the content relevance and the MPP of the user as personalized content items; and
delivering the personalized content items to the user through the user interface as personalized content recommendations.

19. The non-transitory computer-readable storage medium according to claim 18, wherein the content relevance of content items in a curated content pool is determined by:
$${E}_{x , H} = {tf}_{x , H} \times log ( \frac{N}{{df}_{x}} ) ,$$
wherein tf_{x,H} is frequency of a content item x among users H who recovered from a certain mental health issue, N is a total number of the users who were ever diagonalized with the certain mental health issue, and dfₓ is a total number of users used the content item x.
